(19) United States
(12) Patent Application Publication
(10) Pub. No.: US 2025/0320436 A1
(43) Pub. Date: Oct. 16, 2025

(54) CONSUMER PRODUCTS CONTAINING MALODOR COUNTERACTING COMPOUNDS

(71) Applicant: The Procter & Gamble Company, Cincinnati, OH (US)

(72) Inventors: Jennifer Lea RINKER, Hamilton, OH (US); Sylvain Andre SORIN, Waterloo (BE); Gary Bernard WOMACK, Plainsboro, NJ (US); Huda A. JERRI, Plainsboro, NJ (US); Sabine Emmanuelle VON TSCHARNER, Plainsboro, NJ (US); Robert Anton KNAB, Plainsboro, NJ (US); Guillaume Daniel Gabriel LEVADE, Plainsboro, NJ (US)

(21) Appl. No.: 19/036,102

(22) Filed: Jan. 24, 2025

Related U.S. Application Data
(60) Provisional application No. 63/625,318, filed on Jan. 26, 2024.

(51) Int. Cl. C11D 3/50; C11D 3/20

(52) U.S. Cl. CPC C11D 3/50; C11D 3/2086; C11D 2111/12; C11D 2111/14

(57) ABSTRACT

The present invention relates to consumer products that include compounds selected from the group consisting of a compound of formula (I), a compound of formula (II), a compound of formula (III), a compound of formula (IV), and mixtures thereof.

### TECHNICAL FIELD

The present invention relates to compounds selected from the group consisting of a compound of formula (I), a compound of formula (II), a compound of formula (III) and a compound of formula (IV). Furthermore, the present invention relates to the use of said compounds as malodor counteracting ingredients, as well as the perfuming and/or malodor counteracting compositions or consumer products comprising the invention's compounds.

### BACKGROUND

Consumers often correlate the efficiency of perfumed laundry care articles with the long-lastingness or substantivity of perfume perception and seek to have laundry smelling fresh and clean. However, malodors may be perceived on washed garments. Indeed, sweat and body malodor present on dirty garments are often difficult to remove by washing. In addition, malodor may be generated during drying or storage of the garments, such as moldy malodor caused by a humid environment. Malodors are frequently complex mixtures of more than one malodorant compound which may typically include various amines, thiols, sulfides, short chain aliphatic and olefinic acids, e.g., fatty acids, and derivatives thereof.

Malodor counteracting technology may suffer from having a bad impact on the organoleptic properties of the laundry care article and/or on the hedonics experienced during washing or drying steps.

In order to improve the consumer experience and to avoid or limit the presence of malodors on garments, such as moldy malodors, there is a constant need for malodor counteracting ingredients allowing decreasing, blocking or suppressing the perception of malodors without trade-off on the organoleptic impression perceived along the entire laundry process.

Therefore, it is the goal of the present invention to provide compounds and compositions allowing to counteract moldy malodors and to deliver a long-lasting or substantive odor while imparting targeted organoleptic properties to the laundry care articles and during the washing process.

### DESCRIPTION OF THE INVENTION

It has now been discovered that the compounds selected from the group consisting of a compound of formula (I), a compound of formula (II), a compound of formula (III) and a compound of formula (IV) can advantageously be employed as a malodor counteracting technology/ingredient against moldy malodor while bringing a long-lasting or substantive odor from a given surface into the environment by releasing respectively a formate ester selected from the group consisting of a compound of formula (V), a compound of formula (VI), a compound of formula (VII) and a compound of formula (VIII) and/or an alcohol selected from the group consisting of a compound of formula (IX), a compound of formula (X), a compound of formula (XI) and a compound of formula (XII) together with para-methyacetophenone from a given surface into the surrounding environment upon exposure to an environment wherein they are oxidized.

Therefore, a first object of the present invention is a compound selected from the group consisting of a compound of formula (I), a compound of formula (II), a compound of formula (III) and a compound of formula (IV)

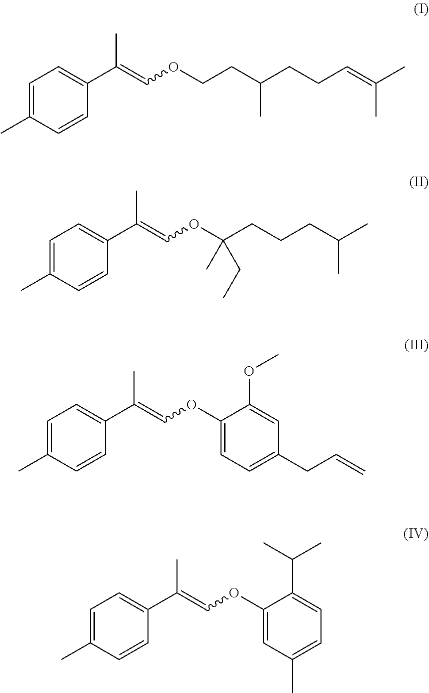

or in the form of any one of their stereoisomers or mixtures thereof.

The compounds of the present invention are surprisingly found to possess unexpected advantages in malodor counteracting applications such as body perspiration, environmental odor such as mold and mildew, bathroom, and etc. The invention's compounds substantially eliminate the perception of malodors and/or prevent the formation of such malodors, thus, can be utilized with a vast number of functional products. Malodor counteracting effective amount is understood to mean the amount of the inventive malodor counteractant employed in a functional product that is organoleptically effective to abate a given malodor while reducing the combined intensity of the odor level, wherein the given malodor is present in air space or has deposited on a substrate. The exact amount of malodor counteractant agent employed may vary depending upon the type of malodor counteractant, the type of the carrier employed, and the level of malodor counteraction desired. In general, the amount of malodor counteractant agent present is the ordinary dosage required to obtain the desired result. Such dosage is known to the skilled practitioner in the art. In a preferred embodiment, when used in conjunction with scented solid or liquid functional products, e.g., soap and detergent, the compounds of the present invention may be present in an amount ranging from about 0.005 to about 50 weight percent, preferably from about 0.01 to about 20 weight percent, preferably from about 0.01 to about 5 weight percent, and more preferably from about 0.01 to about 1 weight percent and when used in conjunction with scented gaseous functional products, the compounds of the present invention may be present in an amount ranging from about 0.1 to 10 mg per cubic meter of air.

The term “malodor counteracting ingredient” or similar is understood as being capable of reducing the perception of malodor, i.e., of an odor that is unpleasant or offensive to the human nose.

For the sake of clarity, by the expression “any one of their stereoisomers or mixtures thereof”, or the similar, it is meant the normal meaning understood by a person skilled in the art, i.e., that the compounds of formula (I) to (IV) can be a pure enantiomer or a diastereoisomer. In other words, the compounds of formula (I) to (IV) possess stereocenters and double bonds and the stereocenters can have two different stereochemistries (e.g., R or S). The compounds of formula (I) to (IV) may even be in the form of a pure enantiomer or in the form of a mixture of enantiomers or diastereoisomers. The compounds of formula (I) to (IV) can be in a racemic or scalemic form. Therefore, the compounds of formula (I) to (IV) can be one stereoisomer or in the form of a composition of matter comprising, or consisting of, various stereoisomers.

For the sake of clarity, by the wavy bond in compounds of formula (I), (II), (III) and (IV), it is meant the normal meaning understood by a person skilled in the art, i.e., that the double bond may have a cis configuration corresponding to the Z isomer, a trans configuration corresponding to the E isomer or a mixture thereof. In other words, the compounds of formula (I), (II), (III) and (IV), may be in the form of its E or Z isomer or of a mixture thereof, e.g., a composition of matter consisting of one or more compounds of formula (I), (II), (III) and (IV), having the same chemical structure but differing by the configuration of the double bond. In particular, the compounds (I), (II), (III) and (IV), can be in the form of a mixture consisting of isomers E and Z and wherein said isomer E represents at least 25% of the total mixture, at least 35%, at least 50%, or even at least 75% (i.e., a mixture E/Z comprised between 75/25 and 100/0). In particular, the double bond of the compounds of formula (I), (II), (III) and (IV) is in E configuration.

Unless specified otherwise, all percentages refer to percent by weight, based on the total weight of the referenced composition.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

According to any embodiments of the invention, the compound of formula (I) is 1-(1-((3,7-dimethyloct-6-en-1-yl)oxy) prop-1-en-2-yl)-4-methylbenzene or (E)-1-(1-((3,7-dimethyloct-6-en-1-yl)oxy) prop-1-en-2-yl)-4-methylbenzene.

According to any embodiments of the invention, the compound of formula (II) is 1-(1-((3,7-dimethyloctan-3-yl)oxy) prop-1-en-2-yl)-4-methylbenzene or (E)-1-(1-((3,7-dimethyloctan-3-yl)oxy) prop-1-en-2-yl)-4-methylbenzene

According to any embodiments of the invention, the compound of formula (III) is 4-allyl-2-methoxy-1-((2-(p-tolyl) prop-1-en-1-yl)oxy)benzene or (E)-4-allyl-2-methoxy-1-((2-(p-tolyl) prop-1-en-1-yl)oxy)benzene.

According to any embodiments of the invention, the compound of formula (IV) is 1-isopropyl-4-methyl-2-((2-(p-tolyl) prop-1-en-1-yl)oxy)benzene or (E)-1-isopropyl-4-methyl-2-((2-(p-tolyl) prop-1-en-1-yl)oxy)benzene.

According to any of the embodiments, the compounds of formula (I), (II), (III) and (IV) are non-volatile and essentially odorless. At the same time, they are relatively stable in perfuming compositions or perfumed consumer product.

Non-volatile and essentially odorless compounds are advantageously characterized by a vapor pressure below 2.0 Pa, as obtained by calculation using the software EPIwin v. 3.10 (2000, available at the US Environmental Protection Agency). Preferably, said vapor pressure is below 0.2 Pa, or preferably below 0.02 Pa, or even more preferably below 0.01 Pa.

The compound according to formula (I), is capable of releasing, via a decomposition reaction para-methylacetophenone together with a formate ester of formula (V) and an alcohol of formula (IX)

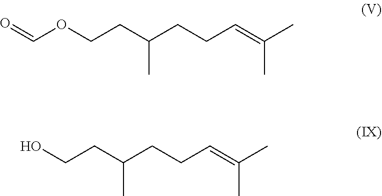

in the form of any one of their stereoisomers or mixtures thereof.

The compound according to formula (III), is capable of releasing, via a decomposition reaction para-methylacetophenone together with a formate ester of formula (VI) and an alcohol of formula (X).

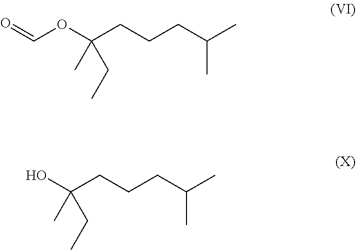

in the form of any one of theirs stereoisomers or mixtures thereof.

The compound according to formula (III), is capable of releasing, via a decomposition reaction para-methylacetophenone together with a formate ester of formula (VII) and an alcohol of formula (XI).

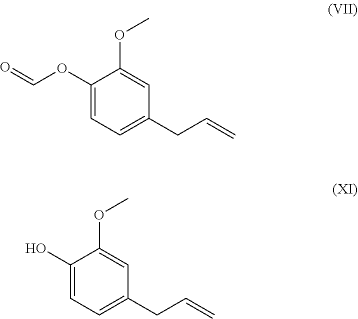

The compound according to formula (IV), is capable of releasing, via a decomposition reaction para-methylacetophenone together with a formate ester of formula (VIII) and an alcohol of formula (XII).

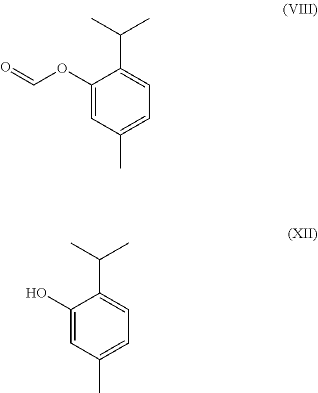

The decomposition reaction, which leads to the release of the compounds as mentioned here-below, is believed to be triggered by oxidation. Indeed, the compounds selected from the group consisting of a compound of formula (I), a compound of formula (II), a compound of formula (III) and a compound of formula (IV) are oxidized under ambient conditions and in absence of any catalyst. For the sake of clarity, by the expression “ambient conditions”, or the similar, it is meant the normal meaning understood by a person skilled in the art, i.e. the oxidation occurs at room temperature, under air and atmospheric pressure. In other words, the environment wherein the compounds are oxidized is air. Herewith it is understood, that the compounds selected from the group consisting of a compound of formula (I), a compound of formula (II), a compound of formula (III) and a compound of formula (IV) are oxidized in ambient air. In particular, it is understood that the compounds selected from the group consisting of a compound of formula (I), a compound of formula (II), a compound of formula (III) and a compound of formula (IV) do not require a pure oxygen environment, heat or catalyst to be oxidized.

According to any of the embodiments, the released compounds as mentioned here-below are advantageously characterized by a vapor pressure above 0.5 Pa, even above 1.0 Pa, as obtained by calculation using the software EPIwin v. 3.10 (2000, available at the US Environmental Protection Agency). According to another embodiment, said vapor pressure is above 5.0, or even above 7.0 Pa.

The invention's compounds of formula (I) to (IV) are delivery systems able to release under certain conditions para-methylacetophenone and respectively compounds of formula (V) to (VIII) and/or compounds of formula (IX) to (XII).

Another object of the present invention is the use of the above-described compounds of formula (I), (II), (III), (IV) and mixtures thereof as delivery system to release perfuming compounds; i.e. use of compounds of formula (I), (II), (III), (IV) as defined above or mixtures thereof as perfuming ingredient to provide a long-lasting odor/effect and a malodor counteracting effect. In other words, it concerns a method to confer, enhance, improve or modify the odor properties of a perfuming composition or the air surrounding the perfuming composition, which method comprises adding to said composition an effective amount of at least one compound selected from the group consisting of compounds of formula (I), (II), (III), (IV), as defined above. By “use of an invention's compound” it has to be understood here also the use of any composition containing said compounds and which can be advantageously employed in the perfumery industry to provide a long-lasting odor/effect and a malodor counteracting effect.

The invention's compound allows counteracting moldy malodor while providing a long-lasting odor effect.

For sake of clarity, a long-lasting effect is typically achieved if, after a certain time, e.g. after several hours or days, a given compound emits higher amounts of an odor into the environment than a reference compound.

Another object of the present invention is a perfuming and/or malodor counteracting composition comprising:
i) at least one compound selected from the group consisting of compounds of formula (I), (II), (III), (IV), as defined above;
ii) at least one perfuming ingredient and/or at least one perfumery carrier; and
iii) optionally at least one perfumery adjuvant.

According to any embodiment of the inventions, at least one compound selected from the group consisting of compound of formula (I), compound of formula (II), compound of formula (III) and compound of formula (IV) can be added to the perfuming and/or malodor counteracting composition in a large range of concentrations. As non-limiting examples, one can cite for at least one compound selected from the group consisting of compound of formula (I), compound of formula (II), compound of formula (III) and compound of formula (IV) concentration values ranging from 0.001% to 50% by weight, or even more, of the compounds of the invention based on the weight of the perfuming and/or malodor counteracting composition into which they are incorporated. Preferably, the at least one compound selected from the group consisting of compound of formula (I), compound of formula (II), compound of formula (III) and compound of formula (IV) concentration will be comprised between from 0.01% to 25% by weight, or even more, of the compounds of the invention based on the weight of the perfuming and/or malodor counteracting composition into which they are incorporated. Preferably, the at least one compound selected from the group consisting of compound of formula (I), compound of formula (II), compound of formula (III) and compound of formula (IV) concentration will be comprised between from 0.01% to 10% by weight, or even more, of the compounds of the invention based on the weight of the perfuming and/or malodor counteracting composition into which they are incorporated. Even more preferably, the at least one compound selected from the group consisting of compound of formula (I), compound of formula (II), compound of formula (III) and compound of formula (IV) concentration will be comprised between from 0.1% to 5% by weight, or even more, of the compounds of the invention based on the weight of the perfuming and/or malodor counteracting composition into which they are incorporated. It goes without saying that the invention's perfuming and/or malodor counteracting composition works also with more of at least one compound selected from the group consisting of compound of formula (I), compound of formula (II), compound of formula (III) and compound of formula (IV). However, the optimum concentration of the invention's compound will depend, as the person skilled in the art knows, on the nature of the latter, on the nature of the perfumery carrier and the perfuming ingredient and optionally the perfumery carrier, on the effect sought.

By “perfumery carrier” it is meant here a material which is practically neutral from a perfumery point of view, i.e., that does not significantly alter the organoleptic properties of perfuming ingredients. Said carrier may be a liquid or a solid.

As liquid carrier one may cite, as non-limiting examples, an emulsifying system, i.e., a solvent and a surfactant system, or a solvent commonly used in perfumery. A detailed description of the nature and type of solvents commonly used in perfumery cannot be exhaustive. However, one can cite as non-limiting examples, solvents such as butylene or propylene glycol, glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, Abalyn® (rosin resins, available from Eastman), benzyl benzoate, benzyl alcohol, 2-(2-ethoxyethoxy)-1-ethano, tri-ethyl citrate or mixtures thereof, which are the most commonly used or also naturally derived solvents like glycerol or various vegetable oils such as palm oil, sunflower oil or linseed oil. For the compositions which comprise both a perfumery carrier/and a perfuming ingredient, other suitable perfumery carriers than those previously specified, can be also ethanol, water/ethanol mixtures, limonene or other terpenes, isoparaffins such as those known under the trademark Isopar (origin: Exxon Chemical) or glycol ethers and glycol ether esters such as those known under the trademark Dowanol (origin: Dow Chemical Company), or hydrogenated castor oils such as those known under the trademark Cremophor RH 40 (origin: BASF).

Solid carrier is meant to designate a material to which the perfuming and/or malodor counteracting composition or some element of the perfuming and/or malodor counteracting composition can be chemically or physically bound. In general, such solid carriers are employed either to stabilize the composition, or to control the rate of evaporation of the compositions or of some ingredients. Solid carriers are of current use in the art and a person skilled in the art knows how to reach the desired effect. However, by way of non-limiting example of solid carriers, one may cite absorbing gums or polymers or inorganic material, such as porous polymers, cyclodextrins, dextrins, maltodextrins, wood-based materials, organic or inorganic gels, clays, gypsum, calcium carbonate, silicon dioxide, talc or zeolites.

As other non-limiting examples of solid carriers, one may cite encapsulating materials. Examples of such materials may comprise wall-forming and plasticizing materials, such as glucose syrups, natural or modified starches, hydrocolloids, cellulose derivatives, polyvinyl acetates, polyvinylalcohols, proteins or pectins, plant gums such as acacia gum (Gum Arabic), urea, sodium chloride, sodium sulfate, sodium carbonate, sodium bicarbonate, calcium carbonate, magnesium sulfate, calcium sulfate, magnesium oxide, zinc oxide, titanium dioxide, calcium chloride, potassium chloride, magnesium chloride, zinc chloride, carbohydrates, saccharides such as sucrose, mono-, di-, tri- and polysaccharides and derivatives such as chitosan, starch, cellulose, carboxymethyl methylcellulose, methylcellulose, hydroxyethyl cellulose, ethyl cellulose, propyl cellulose, polyols/sugar alcohols such as sorbitol, maltitol, xylitol, erythritol and isomalt, polyethylene glycol (PEG), polyvinyl pyrrolidone (PVP), polyvinyl alcohol, acrylamides, acrylates, polyacrylic acid and related structures, maleic anhydride copolymers, amine-functional polymers, vinyl ethers, styrenes, polystyrenesulfonates, vinyl acids, ethylene glycol-propylene glycol block copolymers, pectins, xanthanes, alginates, carragenans, citric acid or any water soluble solid acid, fatty alcohols or fatty acids and mixtures thereof, or yet the materials cited in reference texts such as H. Scherz, Hydrokolloide: Stabilisatoren, Dickungs-und Geliermittel in Lebensmitteln, Band 2 der Schriftenreihe Lebensmittelchemie, Lebensmittelqualität, Behr's Verlag GmbH & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycouryl, melamine, methylol melamine, methylated methylol melamine, guanazole and the like, as well as mixtures thereof. Alternatively, one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Other resins are those produced by the polycondensation of an a polyol, like glycerol, and a polyisocyanate, like a trimer of hexamethylene diisocyanate, a trimer of isophorone diisocyanate or xylene diisocyanate or a Biuret of hexamethylene diisocyanate or a trimer of xylene diisocyanate with trimethylolpropane (known with the tradename of Takenate®, origin: Mitsui Chemicals), among which a trimer of xylene diisocyanate with trimethylolpropane and a Biuret of hexamethylene diisocyanate are preferred.

Some of the seminal literature related to the encapsulation of perfumes by polycondensation of amino resins, namely melamine-based resins with aldehydes includes articles such as those published by K. Dietrich et al. Acta Polymerica, 1989, Vol. 40, pages 243, 325 and 683, as well as 1990, Vol. 41, page 91. Such articles already describe the various parameters affecting the preparation of such core-shell microcapsules following prior art methods that are also further detailed and exemplified in the patent literature. US 4′396′670, to the Wiggins Teape Group Limited, is a pertinent early example of the latter. Since then, many other authors have enriched the literature in this field and it would be impossible to cover all published developments here, but the general knowledge in encapsulation technology is very significant. More recent publications of pertinence, which disclose suitable uses of such microcapsules, are represented for example by the article of K. Bruyninckx and M. Dusselier, ACS Sustainable Chemistry & Engineering, 2019, Vol. 7, pages 8041-8054.

The term “perfuming ingredient” is understood as a compound which is used, for the primary purpose, as an active ingredient in perfuming preparations or compositions in order to impart a hedonic effect. In other words, a compound to be considered as being a perfuming ingredient, must be recognized by a skilled person in the art of perfumery as being able to impart or modify in a positive or pleasant way the odor of a composition, and not just as having an odor. The perfuming ingredient may impart an additional benefit beyond that of modifying or imparting an odor, such as long-lastingness, blooming, malodor counteraction, a cosmetic effect, an antimicrobial effect, an antiviral effect, microbial stability, or pest control. The perfuming ingredient is not a compound according to the invention.

The nature and type of the perfuming ingredients do not warrant a more detailed description here, which in any case would not be exhaustive, the skilled person being able to select them on the basis of general knowledge and according to intended use or application and the desired organoleptic effect. In general terms, these perfuming ingredients belong to chemical classes as va]ried as alcohols, lactones, aldehydes, ketones, esters, ethers, acetates, nitriles, thiols, terpene hydrocarbons, nitrogenous or sulfurous heterocyclic compounds and essential oils, and the perfuming ingredients can be of natural or synthetic origin. In particular, one may cite perfuming ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0^{2,7}]undecan-4-one, 1-methoxy-3-hexanethiol, 2-ethyl-4,4-dimethyl-1,3-oxathiane, 2,2,7/8,9/10-tetramethylspiro[5.5]undec-8-en-1-one, menthol and/or alpha-pinene;
Balsamic ingredients: ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4 (8)-p-menthadiene; Floral ingredients: methyl dihydrojasmonate, linalool, citronellol, phenylethanol, 3-(4-tert-1-butylphenyl)-2-methylpropanal, benzyl acetate, benzyl salicylate, tetrahydro-2-isobutyl-4-methyl-4 (2H)-pyranol, beta ionone, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl) propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl(S)-2-(1,1-dimethylpropoxy) propanoate, 2,2,2-trichloro-1-phenylethyl acetate, 4/3-(4-hydroxy-4-methylpentyl)-3-cyclohexene-1-carbaldehyde, 8-decen-5-olide, 4-phenyl-2-butanone, isononyle acetate, 4-(1,1-dimethylethyl)-1-cyclohexyl acetate, verdyl isobutyrate and/or mixture of methylionone isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl) propanal, diethyl 1,4-cyclohexanedicarboxylate, 3-methyl-2-hexen-1-yl acetate, oct-2-en-4-one, 1-[3,3-dimethylcyclohexylethyl[3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl(2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 4,6,6,7,8,8-hexamethyl-1,3,4,6,7,8-hexahydrocyclopenta[g]isochromene, (1S,1′R)-2-[1-(3′,3′-dimethyl-1′-cyclohexyl) ethoxy]-2-methylpropyl propanoate, oxacyclohexadecan-2-oneand/or (1S,1′R)-[1-(3′,3′-dimethyl-1′-cyclohexyl) ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.02,7]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl) ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3 (4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonan and/or 3-(3-isopropyl-1-phenyl) butanal.

A composition according to the invention may not be limited to the above-mentioned perfuming ingredients, and many other of these ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said ingredients may also be compounds known to release in a controlled manner various types of perfuming compounds also known as properfumes or profragrances. Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, 3-(dodecylthio)-1-(2,6,6-trimethyl-3-cyclohexen-1-yl)-1-butanone, 3-(dodecylsulfonyl)-1-(2,6,6-trimethylcyclohex-3-en-1-yl) butan-1-one, a linear polysiloxane co-polymer of (3-mercaptopropyl)(methyl)dimethoxysilane, 3-(dodecylthio)-1-(6-ethyl-2,6-dimethylcyclohex-3-en-1-yl) butan-1-one, 2-(dodecylthio) octan-4-one, 2-(dodecylsulfonyl) octan-4-one, 4-oxooctan-2-yl dodecanoate, 2-phenylethyl oxo (phenyl)acetate, 3,7-dimethylocta-2,6-dien-1-yl oxo (phenyl)acetate, (Z)-hex-3-en-1-yl oxo (phenyl)acetate, 3,7-dimethyl-2,6-octadien-1-yl hexadecanoate, bis(3,7-dimethylocta-2,6-dien-1-yl) succinate, (2E,6Z)-nona-2,6-dien-1-yl tetradecanoate, (2E,6Z)-nona-2,6-dien-1-yl dodecanoate, (2E,6Z)-nona-2,6-dien-1-yl hexadecanoate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy) ethoxy)benzene, 2-methyl-1-(octan-3-yloxy) undec-1-ene, 1-methoxy-4-(1-phenethoxyprop-1-en-2-yl)benzene, 1-methyl-4-(1-phenethoxyprop-1-en-2-yl)benzene, (2-phenethoxyvinyl)benzene, (2-((2-pentylcyclopentylidene) methoxy)ethyl)benzene, 4-allyl-2-methoxy-1-((2-methoxy-2-phenylvinyl)oxy)benzene, (2-((2-heptylcyclopentylidene) methoxy)ethyl)benzene, 1-methoxy-4-(1-phenethoxyprop-1-en-2-yl)benzene, (2-((2-methyl-4-(2,6,6-trimethylcyclohex-1-en-1-yl) but-1-en-1-yl)oxy)ethyl)benzene, 1-methoxy-4-(2-methyl-3-phenethoxyallyl)benzene, (2-((2-isopropyl-5-methylcyclohexylidene) methoxy)ethyl)benzene, 1-isopropyl-4-methyl-2-((2-pentylcyclopentylidene) methoxy)benzene, 2-methoxy-1-((2-pentylcyclopentylidene) methoxy)-4-propylbenzene, 3-methoxy-4-((2-methoxy-2-phenylvinyl)oxy)benzaldehyde, 1-isopropyl-2-((2-methoxy-2-phenylvinyl)oxy)-4-methylbenzene, 4-((2-(hexyloxy)-2-phenylvinyl)oxy)-3-methoxybenzaldehyde, 3-methyl-5-phenylpentyl hexadecanoate, 3-(dodecylthio)-2-methyl-1-(2,6,6-trimethylcyclohex-3-en-1-yl) butan-1-one, 3,5-bis(1-(4-isopropylphenyl) propan-2-3,5-di(undecan-2-yl)dihydro-1H,3H,5H-yl)dihydro-1H,3H,5H-oxazolo[3,4-c]oxazole, oxazolo[3,4-c]oxazole, 3,5-bis(2,4-dimethylcyclohex-3-en-1-yl)dihydro-1H,3H,5H-oxazolo[3,4-c]oxazole, ethyl 2-acetyl-4-methyltridec-2-enoate, dec-9-en-1-yl(E)-3-(2-hydroxyphenyl) acrylate, 4-(dodecylthio)-4-methylpentan-2-one, methyl or ethyl N,S-bis(4-oxo-4-(2,6,6-trimethylcyclohex-3-en-1-yl) butan-2-yl)-L-cysteinate, 1-butoxy-3-((1E,4Z)-hepta-1,4-dien-1-yl)benzene, 2-methoxy-4-((1E,4Z)-hepta-1,4-dien-1-yl)phenol, 2-ethoxy-4-((1E,4Z)-hepta-1,4-dien-1-yl)phenol, 1-methoxy-4-(3-phenylprop-1-en-1-yl)benzene or mixtures thereof.

In a particular embodiment, the perfuming and/or malodor counteracting composition according to the invention comprises a perfumery adjuvant.

The term “perfumery adjuvant” is understood as an ingredient capable of imparting additional added benefit such as a color, a particular light resistance, chemical stability and etc. A detailed description of the nature and type of adjuvant commonly used in perfuming and/or malodor counteracting compositions cannot be exhaustive, but it has to be mentioned that the ingredients are well known to a person skilled in the art. However, one may cite as specific non-limiting examples the following: viscosity agents (e.g. surfactants, thickeners, gelling and/or rheology modifiers), stabilizing agents (e.g. preservatives, antioxidants, heat/light and or buffers or chelating agents, such as BHT), coloring agents (e.g. dyes and/or pigments), preservatives (e.g. antibacterial or antimicrobial or antifungal or anti-irritant agents), abrasives, skin cooling agents, fixatives, insect repellants, ointments, vitamins and mixtures thereof. By “fixative” also called “modulator”, it is understood here an agent having the capacity to affect the manner in which the odor, and in particular the evaporation rate and intensity, of the compositions incorporating said modulator can be perceived by an observer or user thereof, over time, as compared to the same perception in the absence of the modulator. In particular, the modulator allows prolonging the time during which their fragrance is perceived. Non-limiting examples of suitable modulators may include methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10; isoceteth-12; isoceteth-15; isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, n-hexadecyl n-nonanoate, noctadecyl n-nonanoate, cyclodextrin, and a combination thereof. At most 20% by weight, preferably at most 10% by weight, preferably at most 5% by weight, even more preferably at most 3%, based on the total weight of the perfuming and/or malodor counteracting composition, of the modulator may be incorporated into the perfumed consumer product.

It is understood that a person skilled in the art is perfectly able to design optimal formulations for the desired effect by admixing the above-mentioned components of a perfuming and/or malodor counteracting composition, simply by applying the standard knowledge of the art as well as by trial-and-error methodologies.

An invention's composition consisting of at least one compounds of the invention and at least one perfumery carrier consists of a particular embodiment of the invention as well as a perfuming and/or malodor counteracting composition comprising at least one compound of the invention, at least one perfumery carrier, at least one perfuming ingredient, and optionally at least one perfumery adjuvant.

According to a particular embodiment, the compositions mentioned above, comprise more than one invention's compounds and enable the perfumer to prepare accords or perfumes possessing the long-lasting odor tonality of various compounds released by the invention's compounds, creating thus new building block for creation purposes.

For the sake of clarity, it is also understood that any mixture resulting directly from a chemical synthesis, e.g. a reaction medium without an adequate purification, in which the compound of the invention would be involved as a starting, intermediate or end-product could not be considered as a perfuming and/or malodor counteracting composition according to the invention as far as said mixture does not provide the inventive compound in a suitable form for perfumery. Thus, unpurified reaction mixtures are generally excluded from the present invention unless otherwise specified.

Another object of the present invention consists of a consumer product comprising a compound selected from the group consisting of a compound of formula (I), a compound of formula (II), a compound of formula (III), a compound of formula (IV), and mixtures thereof

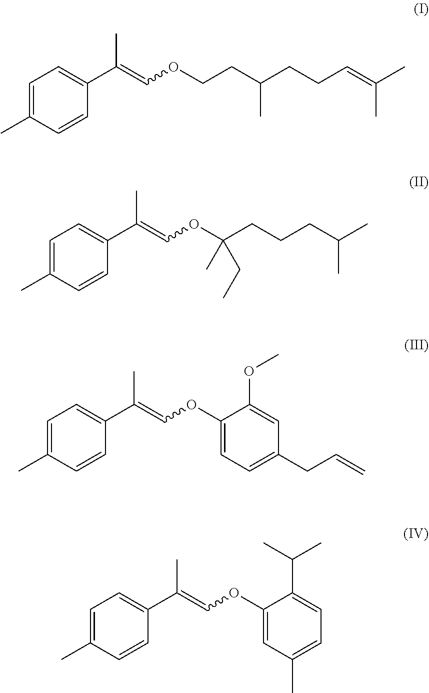

or in the form of any one of its stereoisomers, or mixtures thereof.

The invention's compounds can be added as such or as part of an invention's perfuming and/or malodor counteracting composition.

As used herein “consumer product” means baby care, beauty care, fabric and home care, family care, feminine care or health care products generally intended to be used or consumed in the form in which it is sold.

“Baby care” includes but are not limited to products and/or methods relating to disposable absorbent and/or non-absorbent articles including adult incontinence garments, bibs, diapers, training pants, infant and toddler care wipes; and personal care products, including hand soaps, shampoos, lotions, oral care implements, and clothing.

“Beauty care” includes products for and/or methods relating to treating hair (human, dog, and/or cat), including, bleaching, coloring, dyeing, conditioning, growing, removing, retarding growth, shampooing, styling; deodorants and antiperspirants; personal cleansing; color cosmetics; products, and/or methods relating to treating skin (human, dog, and/or cat), including application of creams, lotions, and other topically applied products for consumer use; and products and/or methods relating to orally administered materials for enhancing the appearance of hair, skin, and/or nails (human, dog, and/or cat); and shaving.

“Fabric and home care” includes products for and/or methods relating to treating fabrics, hard surfaces and any other surfaces in the area of fabric and home care, including: air care, car care, dishwashing, fabric conditioning (including softening), laundry detergency, laundry and rinse additive and/or care, hard surface cleaning and/or treatment, and other cleaning for consumer and or institutional use.

“Family care” includes products such as wet or dry bath tissue, facial tissue, disposable handkerchiefs, disposable towels, and/or wipes, as well as methods and equipment for making such products.

“Feminine care” includes products and/or methods relating to catamenial pads, incontinence pads, interlabial pads, panty liners, pessaries, sanitary napkins, tampons and tampon applicators, and/or wipes.

“Health care” includes products and/or methods relating to: oral care including any composition for use with any soft and/or hard tissue of the oral cavity or conditions associated therewith (e.g., anti-caries compositions, anti-microbial compositions, anti-plaque chewing gum compositions, breath compositions, confectionaries, dentifrices, denture compositions, lozenges, rinses, and tooth whitening compositions), cleaning devices, floss and flossing devices, and toothbrushes; over-the-counter health care including cough and cold remedies and treatments for other respiratory conditions, pain relievers whether topical, oral, or otherwise, gastrointestinal remedies including any composition suitable for the alleviation of gastrointestinal conditions such as heartburn, upset stomach, diarrhea, and irritable bowel syndrome, and nutrient supplementation such as calcium or fiber supplementation; pharmaceutical care including pharmacologically active molecular and/or biological entities, their use in the treatment and/or prevention of diseases and/or alleviation of symptoms in humans and/or animals, and formulations, regimens, kits and/or routes of delivering such entities to subjects in need of treatment and/or prevention and/or alleviation, discovery tools including screening methods, assays, and receptors, and their use in discovering new pharmacologically active molecular and/or biological entities; pet health and nutrition including pet foods, treats. other orally deliverable products, regardless of distribution channel (including veterinary and over-the-counter), as well as topical products such as grooming aids, training aids, devices, toys, and diagnostic techniques; and waters including purified, flavored, or other treated waters.

As used herein, the term “cleaning and/or treatment composition” is a subset of consumer products that includes, unless otherwise indicated, beauty care, fabric & home care products. Such products include, but are not limited to, products for treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use; and shaving products, products for treating fabrics, hard surfaces and any other surfaces in the area of fabric and home care, including: air care including air fresheners and scent delivery systems, car care, dishwashing, fabric conditioning (including softening and/or freshening), laundry detergency, laundry and rinse additive and/or care, hard surface cleaning and/or treatment including floor and toilet bowl cleaners, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, dentifrice, car or carpet shampoos, bathroom cleaners including toilet bowl cleaners; hair shampoos and hair-rinses; shower gels, and foam baths and metal cleaners; as well as cleaning auxiliaries such as bleach additives and “stain-stick” or pre-treat types, substrate-laden products such as dryer added sheets, dry and wetted wipes and pads, nonwoven substrates, and sponges; as well as sprays and mists all for consumer or/and institutional use; and/or methods relating to oral care including toothpastes, tooth gels, tooth rinses, denture adhesives, tooth whitening.

As used herein, the term “fabric and/or hard surface cleaning and/or treatment composition” is a subset of cleaning and treatment compositions that includes, unless otherwise indicated, granular or powder-form] all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, car or carpet shampoos, bathroom cleaners including toilet bowl cleaners; and metal cleaners, fabric conditioning products including softening and/or freshening that may be in liquid, solid and/or dryer sheet form; as well as cleaning auxiliaries such as bleach additives and “stain-stick” or pre-treat types, substrate-laden products such as dryer added sheets, dry and wetted wipes and pads, nonwoven substrates, and sponges; as well as sprays and mists. All of such products which were applicable may be in standard, concentrated or even highly concentrated form even to the extent that such products may in certain aspect be non-aqueous. Fabric and/or hard surface cleaning and/or treatment compositions may be configured to be sprayable, pourable, dosable, and the like.

As used herein, the term “solid” includes granular, powder, bar and tablet product forms.

As used herein, the term “fluid” includes liquid, gel, paste and gas product forms.

As used herein, the term “area” is used to reference the location or space to be treated or cleaned with a consumer product. An area may be a substrate, paper product, fabric, garment, hard surface, hair, skin, air, and the like.

According to any embodiments of the invention, the consumer product according to the invention comprises at least one compound selected from the group consisting of a compound of formula (I), a compound of formula (II), a compound of formula (III) and a compound of formula (IV), as well as optional additional benefit agents that correspond to the desired consumer product, e.g. a conditioner, a detergent or an air freshener, and an olfactorily effective amount of the perfuming and/or malodor counteracting composition according to the invention. For the sake of clarity, the consumer product is a non-edible product.

The nature and type of the constituents of the consumer product do not warrant a more detailed description here, which in any case would not be exhaustive, the skilled person being able to select them on the basis of his general knowledge and according to the nature and the desired effect of the product.

According to a particular embodiment, the consumer products detailed herein may be in the form of a beauty care or fabric and home care consumer product comprising ingredients that are common in beauty care, fabric and home care consumer products, in particular shower gels, shampoos, soaps, fabric detergents or softeners and all-purpose cleaners. The main functional constituents of perfumed consumer products are surfactants and/or softener components capable of cleaning and/or softening fabrics and/or textiles of varied nature, such as clothes, curtain fabrics, carpets and furniture fabrics, etc., or other home surfaces, skin or hair, and typically used in a large amount of water or water-based solvents. These are therefore formulations wherein the amount of water may be up to 99% by weight of the perfumed consumer product with the exception of solid product such as soaps, solid scent boosters, dryer sheets or solid detergents, wherein the amount of water is at most 20%.

A more detailed description of such fabric cleaning and/or softening formulations is not warranted here, many descriptions of current liquid formulations can be found in the cleaner/fabric softener's patent and other pertinent literature, such as for example the textbook of Louis Ho Tan Tai, “Détergents et Produits de Soins Corporels, Chapters 1 to 7 in particular, Dunod, Paris, 1999, or any other similar and/or more recent textbooks pertaining to the art of liquid softener and all-purpose cleaners formulations. A patent publication, WO 2010/105873, is also cited by way of example, in as much as it describes typical current ingredients, other than perfumes, of such liquid products, particularly on pages 9 to 21. Of course, many other examples of liquid cleaner and/or fabric softener formulations can be found in the literature. Any such liquid formulations, namely liquid fabric cleaners or conditioners and/or all-purpose cleaners, can be used in the here-described compositions. Other examples of fabric detergents or softener compositions into which the compounds of the invention can be incorporated are described in WO 97/34986 or in U.S. Pat. Nos. 4,137,180 and 5,236,615 or EP 799 885. Other typical detergent and softening compositions which can be used are described in works such as Ullmann's Encyclopedia of Industrial Chemistry, Vol. 20, Wiley-VCH, Weinheim, p. 355-540 (2012); Flick, Advanced Cleaning Product Formulations, Noye Publication, Park Ridge, New Jersey (1989); Showell, in Surfactant Science Series, Vol. 71: Powdered Detergents, Marcel Dekker, New York (1988); Proceedings of the World Conference on Detergents (4th, 1998, Montreux, Switzerland), AOCS print.

According to a particular embodiment of the invention, the consumer product may be a liquid fabric softener comprising at least one compound selected from the group consisting of a compound of formula (I), a compound of formula (II), a compound of formula (III) and a compound of formula (IV) and a fabric softener active base in amount comprised between 85 and 100% by weight, based on the total weight of the perfumed consumer product. The main constituent of the fabric softener active base is water or water-based solvents. The fabric softener active base may comprise dialkyl quaternary ammonium salts, dialkyl ester quaternary ammonium salts, Hamburg esterquat, triethanolamine quat, silicones and mixtures thereof. Optionally, the fabric softener active base may further comprise a viscosity modifier in an amount comprised between 0.05 and 1% by weight, based on the total weight of the liquid base; preferably chosen from the group consisting of calcium chloride.

According to a particular embodiment of the invention, the consumer product may be an all-purpose cleaner comprising at least one compound selected from the group consisting of a compound of formula (I), a compound of formula (II), a compound of formula (III) and a compound of formula (IV) and an all-purpose cleaner active base in amount comprised between 85 and 100% by weight, based on the total weight of the consumer product. The main constituent of the all-purpose cleaner active base is water or water-based solvents. The all-purpose active base may comprise linear alkylbenzene sulfonates (LAS) in an amount comprised between 0 and 4%, preferably 1 and 2%, nonionic surfactant in an amount comprised between 0 and 8%, preferably 2 and 4% and acid such as citric acid in an amount comprised between 0.1 and 0.5%.

According to a particular embodiment of the invention, the consumer product may be a liquid detergent comprising at least one compound selected from the group consisting of a compound of formula (I), a compound of formula (II), a compound of formula (III) and a compound of formula (IV) and liquid detergent active base in amount comprised between 85 and 100% by weight, based on the total weight of the consumer product. The main constituent of the liquid detergent active base active base is water or water-based solvents. The liquid detergent active base may comprise anionic surfactant such as alkylbenzenesulfonate (ABS), linear alkylbenzene sulfonates (LAS), secondary alkyl sulfonate (SAS), primary alcohol sulfate (PAS), lauryl ether sulfate (LES), sodium lauryl ether sulfate (SLES), methyl ester sulfonate (MES); nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxides, alkyl polyglucosides, alkyl polyglucosamides; or mixtures thereof.

According to a particular embodiment of the invention, the consumer product may be a solid detergent comprising at least one compound selected from the group consisting of a compound of formula (I), a compound of formula (II), a compound of formula (III) and a compound of formula (IV) and a solid detergent active base in amount comprised between 85 and 100% by weight, based on the total weight of the consumer product. The solid detergent active base may comprise at least one surfactant chosen from the group consisting of anionic, nonionic, cationic, zwiterionic surfactant and mixtures thereof. The surfactant in the solid detergent active base is preferably chosen from the group consisting of linear alkene benzene sulfonate (LABS), sodium laureth sulfate, sodium lauryl ether sulfate, sodium lauryl sulfate (SLS), alpha olefin sulfonate (AOS), methyl ester sulfonates, alkyl polyglyucosides (APG), primary alcohol ethoxylates and in particular lauryl alcohol ethoxylates (LAE), primary alcohol sulfonates, soap and mixtures thereof. The solid detergent active base may comprise a further component, commonly used in powder detergent consumer product, selected from the group consisting of bleaching agents such as TAED (tetraacetylethylenediamine); buffering agent; builders such as zeolites, sodium carbonate or mixture thereof; soil release or soil suspension polymers; granulated enzyme particles such as cellulase, lipase, protease, mannanase, pectinase or mixtures thereof; corrosion inhibitor; antifoaming; sud suppressing agents; dyes; fillers such as sodium silicate, sodium sulfate or mixture thereof; source of hydrogen peroxide such as sodium percarbonate or sodium perborate; and mixtures thereof.

The use of the compounds of the present invention is widely applicable in current consumer products, including personal care products such as soaps, shower gels, and hair care products, fabric care products, air fresheners, and cosmetic preparations. The present invention can also be used with cleaning agents, such as, but not limited to detergents, dishwashing materials, scrubbing compositions, window cleaners and the like.

The invention's compounds of formula (I) to (IV) and stereoisomers thereof are suitable for use in consumer products at levels, based on total consumer product weight, of from about 0.0001% to about 25%, from about 0.0005% to about 10%, from about 0.001% to about 5%, from about 0.005% to about 2.5%, or even from 0.01% to about 1%. Such invention's compounds of formula (I) to (IV) and stereoisomers thereof may be used in various combinations in the aforementioned consumer products. A consumer product may comprise one or more compounds of formula (I) to (IV) and/or stereoisomers thereof.

The invention's compounds of formula (I) to (IV) and stereoisomers thereof are suitable for use in cleaning and/or treatment compositions at levels, based on total cleaning and treatment products weight of from about 0.0001% to about 25%, from about 0.0005% to about 10%, from about 0.001% to about 5%, from about 0.005% to about 2.5%, or even from 0.01% to about 1%. Such invention's compounds of formula (I) to (IV) and stereoisomers thereof may be used in various combinations in the aforementioned cleaning and/or treatment compositions. A cleaning and/or treatment composition as detailed herein may comprise one or more compounds of formula (I) to (IV) and any/or stereoisomers thereof.

The compounds of formula (I) to (IV) and stereoisomers thereof may be suitable for use in fabric and/or hard surface cleaning and/or treatment compositions at levels, based on total fabric and/or hard surface cleaning and/or treatment composition weight, of from about 0.00001% to about 25%, from 0.00005% to about 10%, from 0.0001% to about 5%, from 0.0005% to about 1.0%, or even from 0.005% to about 1%. Such compounds of formula (I) to (IV) and stereoisomers thereof may be used in various combinations in the aforementioned fabric and/or hard surface cleaning and/or treatment compositions. A fabric and/or hard surface cleaning and/or treatment composition may comprise one or more compounds of formula (I) to (IV) and stereoisomers thereof.

A detergent that may comprise the same level of at least one of the invention's compounds of formula (I) to (IV) as disclosed for the aforementioned fabric and hard surface cleaning and/or treatment compositions is disclosed. A detergent may comprise one or more compounds of Formula (I) to (IV) and stereoisomers thereof selected.

The compounds of formula (I) to (IV) and stereoisomers thereof may be suitable for use in highly compacted consumer products, including highly compacted fabric and hard-surface cleaning and/or treatment compositions. For example, the compounds of formula (I) to (IV) and stereoisomers thereof may be employed in solid or fluid highly compacted detergents at levels of from about 0.00001% to about 25%, from 0.00005% to about 10%, from 0.0001% to about 5%, from 0.0005% to about 1.0%, or even from 0.005% to about 1%, based on total composition weight. Such compounds of formula (I) to (IV) and stereoisomers thereof may be used in various combinations in the aforementioned highly compacted detergent compositions. Such highly compact detergents typically comprise a higher-than-normal percentage of active ingredients. A highly compacted detergent may comprise one or more compounds of formula (I) to (IV) and stereoisomers thereof. More specifically, a highly compacted detergent may comprise one or more compounds of formula (I) to (IV) and stereoisomers thereof.

Some of the consumer products disclosed herein can be used to clean or treat an area, such as a surface or fabric. In one example, at least a portion of the area is contacted with a consumer product, in neat form or diluted in a liquor, for example, a wash liquor and then the area may be optionally washed and/or rinsed. In one aspect, an area is optionally washed and/or rinsed, contacted with the consumer product and then optionally washed and/or rinsed. For purposes of the present invention, washing includes but is not limited to, scrubbing, and mechanical agitation. The fabric may comprise most any fabric capable of being laundered or treated in normal consumer use conditions. Liquors that may comprise the disclosed compositions may have a pH of from about 2 to about 11.5. Such compositions are typically employed at concentrations of from about 500 ppm to about 15,000 ppm in solution. When the wash solvent is water, the water temperature typically ranges from about 5° C. to about 90° C. and, when the area comprises a fabric, the water to fabric ratio is typically from about 1:1 to about 30:1.

The consumer product may also be sprayed onto the area to be treated. In some uses, after spraying, the consumer product may be wiped with a cleaning implement such as a sponge, cloth, towel, or substrate, for example. In some uses, after the area is sprayed with the consumer product, the area may be rinsed with water.

Another aspect of the invention is a method of treating an area with the consumer product as defined above.

Another aspect of the invention is a method to release from a precursor compound, compounds selected from the group consisting of:
a) para-methylacetophenone;
b) a formate ester selected from the group consisting of a compound of formula (V), a compound of formula (VI), a compound of formula (VII) and a compound of formula (VIII)

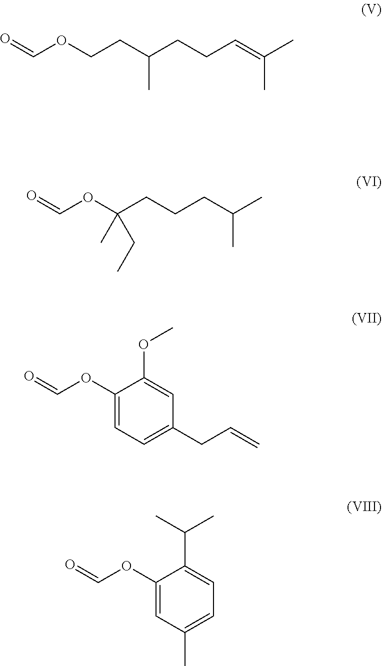

in the form of any one of its stereoisomers or mixtures thereof;
   c) an alcohol selected from the group consisting of a compound of formula (IX), a compound of formula (X), a compound of formula (XI) and a compound of formula (XII)

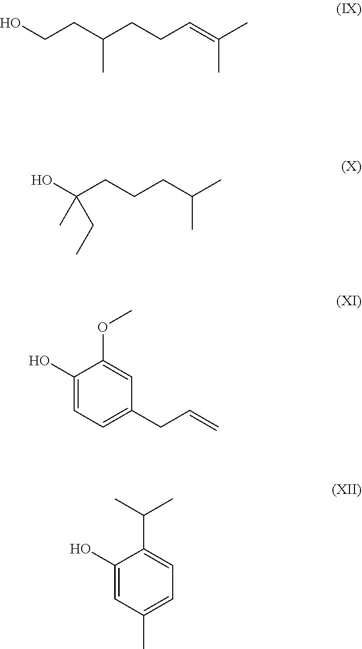

in the form of any one of its stereoisomers or mixtures thereof;

wherein the precursor compound is a compound selected from the group consisting of a compound of formula (I), a compound of formula (II), a compound of formula (III) and a compound of formula (IV)

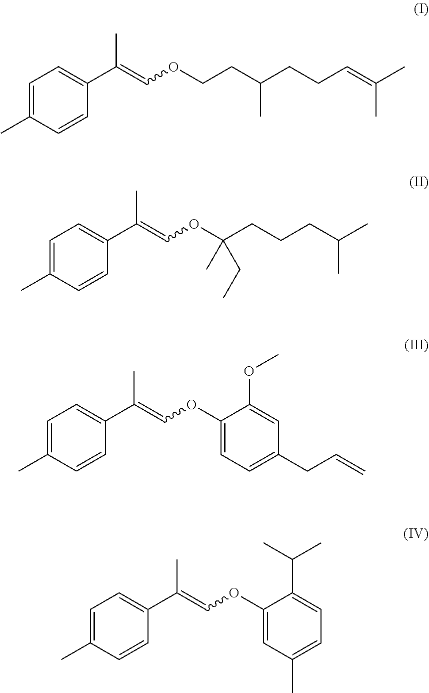

in the form of any one of its stereoisomers or mixtures thereof by exposing the precursor compound to an environment wherein the compound is oxidized.

In a further aspect, the present invention also relates to the use a precursor compound for releasing compounds selected from the group consisting of
a) para-methylacetophenone;
b) a formate ester selected from the group consisting of a compound of formula (V), a compound of formula (VI), a compound of formula (VII) and a compound of formula (VIII)

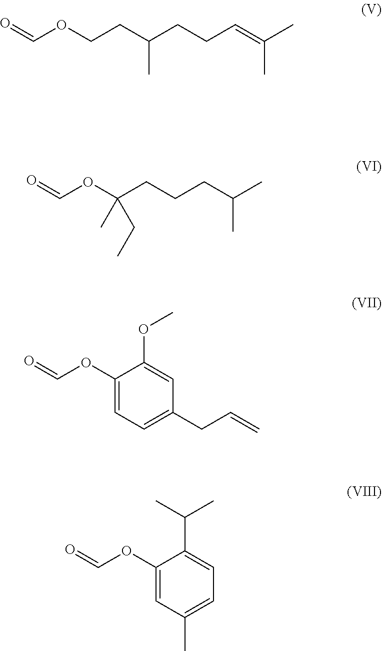

in the form of any one of its stereoisomers or mixtures thereof;
c) an alcohol selected from the group consisting of a compound of formula (IX), a compound of formula (X), a compound of formula (XI) and a compound of formula (XII)

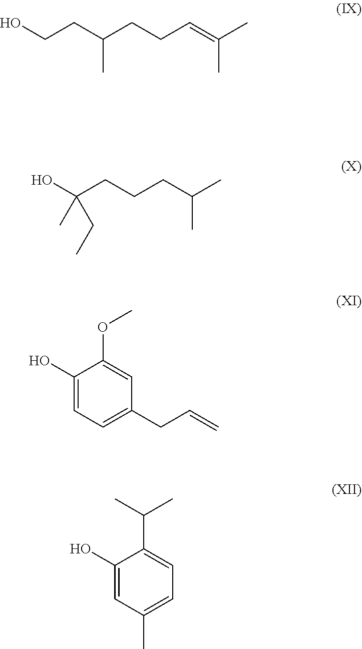

in the form of any one of its stereoisomers or mixtures thereof; wherein the precursor compound cis a compound selected from the group consisting of a compound of formula (I), a compound of formula (II), a compound of formula (III) and a compound of formula (IV)

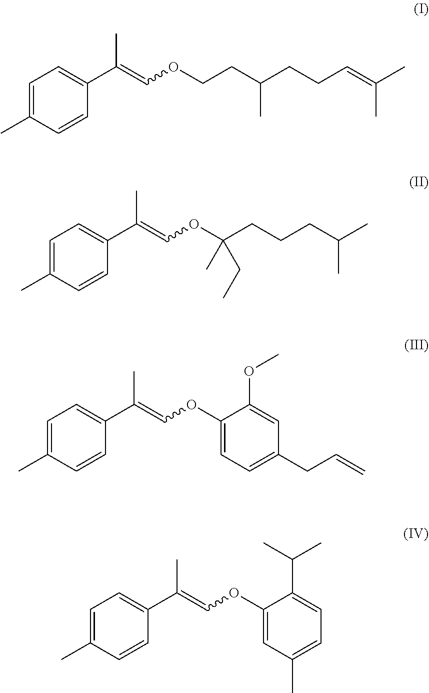

in the form of any one of its stereoisomers, or mixtures thereof by exposing the precursor compound to an environment wherein the compound is oxidized.

Another aspect of the invention is a method for reducing, preventing, or inhibiting the perception of malodor in a subject, the method comprising contacting the subject with a perfuming and/or malodor counteracting composition as defined above or with one or more of a compound selected from the group consisting of a compound of formula (I), a compound of formula (II), a compound of formula (III) and a compound of formula (IV) as defined above, in an amount sufficient to reduce, prevent, or inhibit the subject's perception of malodor.

Another aspect of the invention is a method for intensifying or prolonging the diffusion effect of the characteristic fragrance of para-methylacetophenone, of at least one formate of formula (V), (VI), (VII) and/or (VIIII) and/or of at least one alcohol of formula (IX), (X), (XI) and/or (XII) as defined above, by adding to a perfuming and/or malodor counteracting composition an effective amount of at least one compound selected from the group consisting of a compound of formula (I), a compound of formula (II), a compound of formula (III), a compound of formula (IV) as defined above and subjecting said composition under conditions susceptible of allowing the release of para-methylacetophenone, of at least one formate of formula (V), (VI), (VII) and/or (VIIII) and/or of at least one alcohol of formula (IX), (X), (XI) and/or (XII) over time.

In a further aspect, the present invention relates to the use of at least one compound selected from the group consisting of a compound of formula (I), a compound of formula (II), a compound of formula (III), a compound of formula (IV) as defined above for intensifying or prolonging the diffusion effect, and/or perception of the characteristic fragrance of para-methylacetophenone, of at least one formate of formula (V), (VI), (VII) and/or (VIIII) and/or of at least one alcohol of formula (IX), (X), (XI) and/or (XII) as defined above, by adding to a perfuming and/or malodor counteracting composition an effective amount of at least one compound selected from the group consisting of a compound of formula (I), a compound of formula (II), a compound of formula (III), a compound of formula (IV) as defined above and subjecting said composition under conditions susceptible of allowing the release of para-methylacetophenone, of at least one formate of formula (V), (VI), (VII) and/or (VIIII) and/or of at least one alcohol of formula (IX), (X), (XI) and/or (XII) over time.

In a further aspect, the present invention relates to the use of at least one compound selected from the group consisting of a compound of formula (I), a compound of formula (II), a compound of formula (III), a compound of formula (IV) as defined above to confer, enhance, improve or modify the odor properties of a perfuming composition, the air surrounding the perfuming composition, comprising adding to the composition an effective amount of at least one compound of formula (I) as defined above.

The compounds of formula (I), (II), (III) and (IV) can be prepared according to standard methods known in the art as described herein-below.

### EXAMPLES

The invention is hereafter described in a more detailed manner by way of the following examples, wherein the abbreviations have the usual meaning in the art, temperatures are indicated in degrees centigrade (° C.). NMR spectral data were recorded on a Bruker AMX 500 spectrometer in CDCl₃ at 500 MHz for ¹H and at 125.8 MHz for ¹³C if not indicated otherwise, the chemical displacements □ are indicated in ppm with respect to Si(CH₃)₄ as the standard, the coupling constants J are expressed in Hz (br.=broad peak). Reactions were carried out in standard glassware under N₂. Commercially available reagents and solvents were used without further purification if not stated otherwise.

Although specific conformations or configurations are indicated for some of the compounds, this is not meant to limit the use of these compounds to the isomers described. According to the invention, all possible conformation or configuration isomers are expected to have a similar effect.

Typical manners to prepare the invention's compound and to execute the invention's method are reported herein below in the examples.

### Example 1

Preparation of the invention's compounds of formula (I), (II), (III) and (IV)

Compound of formula (I). 1-(1-((3,7-dimethyloct-6-en-1-yl)oxy) prop-1-en-2-yl)-4-methylbenzene: Part 1 1-(1-methoxyprop-1-en-2-yl)-4-methylbenzene: Methoxymethyltriphenylphosphonium chloride (191.6 g, 559 mmol) and para-methylacetophenone (50.3 g, 375 mmol) were added to 500 ml of toluene. Potassium t-butoxide (66.9 g, 584 mmol) was added to the stirring slurry in 4 portions every 15 min. The mixture was stirred another 3 h becoming a deep red color. It then was poured into 500 ml of water. The mixture was stirred for 15 min and then extracted with EtOAc (3×250 mL). The organic phases were combined, dried over Na₂SO₄, filtered, and concentrated to a grainy solid. The solid was washed with hexane and filtered. Concentrating the filtrate afforded the crude methyl enol ether that was purified by fractional distillation (bp 90° C., 500 mTorr) and isolated as a colorless liquid (56.8 g, 93% yield).

Part 2 1-(1-methoxyprop-1-en-2-yl)-4-methylbenzene: 1-(1-Methoxyprop-1-en-2-yl)-4-methylbenzene (30 g, 185 mmol), (±)-citronellol (57.8 g, 370 mmol) and KHSO₄ (0.25 g, 1.8 mmol) were added to a round-bottomed flask equipped with a distillation head and nitrogen bubbler. The mixture was heated at 150° C. (oil bath) for 1 h while distilling out liberated methanol. The mixture was allowed to cool then placed under vacuum (500-250 mTorr) and heated at 170° C. (oil bath) for 2 h while allowing excess citronellol to distill from the reaction flask. After allowing the mixture to cool, Na₂CO₃ (1 g) was added and the title compound was isolated by distillation from the reaction vessel (bp 130-140° C., 10 mTorr) as a colorless oil (37.2 g, 70% yield, E/Z=80:20).

¹H NMR (CD₂Cl₂, 500 MHz): δ 0.92_{Z} and 0.93_{E} (both d, J=6.8 Hz, 3H), 1.14-1.25 (m, 1H), 1.31-1.41 (m, 1H), 1.42-1.51 (m, 1H), 1.56-1.66 (m, 1H), 1.60 (s, 3H), 1.67 (s, 3H), 1.69-1.75 (m, 1H), 1.88z and 1.94; (both d, ⁴J=1.3 Hz, 3H), 1.95-2.07 (m, 2H), 2.29 (s, 2.4H), 2.30 (s, 0.6H), 3.80-3.91 (m, 2H), 5.08-5.14 (m, 1H), 6.15z and 6.46; (both q, ⁴J=1.3 Hz, 1H), 7.08 (d, J=8.2 Hz, 1.6H), 7.10 (d, J=8.3 Hz, 0.4H), 7.18 (d, J-8.2 Hz, 1.6H), 7.50 (d, J=8.3 Hz, 0.4H).

¹³C NMR (CD₂Cl₂, 125.8 MHz): δ 12.7 (CH₃), 17.7 (CH₃), 18.4 (CH₃), 19.66 (CH₃), 19.71 (CH₃), 21.0 (CH₃), 21.2 (CH₃), 25.81 (CH₃), 25.83 (CH₂), 29.79 (CH), 29.80 (CH), 37.1 (CH₂), 37.2 (CH₂), 37.48 (CH₂), 37.50 (CH₂), 71.3 (CH₂), 71.7 (CH₂), 110.2 (C), 114.0 (C), 125.0 (CH), 125.08 (CH), 125.1 (CH), 127.6 (CH), 128.8 (CH), 129.3 (CH), 131.5 (C), 135.7 (C), 135.73 (C), 136.0 (C), 138.2 (C), 143.7 (CH), 144.0 (CH).

Compound of formula (II). 1-(1-((3,7-dimethyloctan-3-yl)oxy) prop-1-en-2-yl)-4-methylbenzene: A hexane solution (200 ml) of 1-(1-methoxyprop-1-en-2-yl)-4-methylbenzene (10 g, 61.6 mmol), (±)-3,7-dimethyloctan-3-ol (26.8 g, 169 mmol) and pTSA (0.42 g, 2.24 mmol) was refluxed for 6 h. The liberated methanol was removed with a Dean-Stark trap. The reaction mixture was washed with sat. Na₂CO₃. The recovered organic phase was dried with MgSO₄, filtered, and concentrated. Solid Na₂CO₃ (0.5 g) was added to the crude product and the title compound was isolated by distillation (bp 125-130° C., 15 mTorr) as a colorless oil (12.7 g, 72% yield, E/Z=85:15).

¹H NMR (d₆-acetone, 500 MHz): δ 0.86_{Z} and 0.87_{E} (both d, J=6.7 Hz, 6H), 0.87_{Z} and 0.90_{E} (both t, J=7.5 Hz, 3H), 1.16-1.22 (m, 2H), 1.256_{Z} and 1.26_{E} (s, 3H), 1.33-1.43 (m, 2H), 1.51-1.74 (m, 5H), 1.89_{Z} and 1.94_{E} (both d, ⁴J=1.3 Hz, 3H), 2.27_{E} and 2.28_{Z} (both s, 3H), 6.49z and 6.79; (both q, ⁴J=1.3 Hz, 1H), 7.06 (d, J=8.3 Hz, 1.7H), 7.08 (d, J-8.3 Hz, 0.3H), 7.21 (d, J=8.3 Hz, 1.7H), 7.60 (d, J=8.3 Hz, 0.3H).

¹³C NMR (d₆-acetone, 125.8 MHZ): δ 8.2 (CH₃), 8.3 (CH₃), 12.8 (CH₃), 18.7 (CH₃), 20.9 (CH₃), 21.1 (CH₃), 21.86 (CH₂), 21.89 (CH₂), 22.9 (CH₃), 23.3 (CH₃), 23.5 (CH₃), 28.46 (CH), 28.5 (CH), 31.8 (CH₂), 32.0 (CH₂), 39.2 (CH₂), 39.3 (CH₂), 40.15 (CH₂), 40.17 (CH₂), 80.9 (C), 81.4 (C), 110.3 (C), 114.7 (C), 125.4 (CH), 128.1 (CH), 129.0 (CH), 129.6 (CH), 135.3 (C), 135.4 (C), 137.0 (C), 137.9 (CH), 138.1 (CH), 139.3 (C).

Compound of formula (III). 4-allyl-2-methoxy-1-((2-(p-tolyl) prop-1-en-1-yl)oxy)benzene: Eugenol (50 g, 304.5 mmol), 1-(1-methoxyprop-1-en-2-yl)-4-methylbenzene (74.1 g, 457 mmol) and KHSO₄ (12.4 g, 91.4 mmol) were charged to a round-bottomed flask equipped with a Vigreux column (175 mm) and distillation head. The mixture was placed under vacuum (14 mbar) and heated at 140° C. (oil bath) for 5 h while maintaining the reagents at reflux but allowing MeOH to evaporate from the system. The reaction mixture was allowed to cool, and the pressure reduced to 1 mbar. The mixture then was progressively heated to 140° C. (oil bath) while incrementally reducing the pressure to 0.03 mbar. The mixture was heated an additional 2 h while allowing any unreacted starting materials to distill from the system. The reaction mixture was diluted in diethyl ether and washed with sat. aqueous Na₂CO₃ and water. The aqueous phases were extracted with diethyl ether and the combined organic phases were dried over MgSO₄, filtered, and concentrated. The residue was subjected to bulb-to-bulb distillation (oven 210° C., 20 mTorr) to afford the title compound (55 g, 61% yield) as a pale-yellow oil (E/Z=80:20).

¹H NMR (CD₂Cl₂, 600 MHZ): δ 1.98_{Z} and 2.11_{E} (both d, ⁴J=1.3 Hz, 3H), 2.31_{E} and 2.32_{Z} (both s, 3H), 3.35 (d, J=6.8 Hz, 2H), 3.81Z and 3.85_{E} (both s, 3H), 5.06 (dq, J=10.1, 1.3 Hz, 1H), 5.09 (dq, J=16.9, 1.6 Hz, 1H), 5.96 (ddt, J=16.9, 10.1, 6.8 Hz, 1H), 6.40z (q, 4J=1.3 Hz, 0.2H), 6.70-6.74 (m, 1.8H), 6.77z and 6.79; (both d, J=1.8 Hz, 1H), 6.93; and 6.98z (both d, J=8.1 Hz, 1H), 7.10-7.16 (m, 2H), 7.26; and 7.52z (both d, J-8.2 Hz, 2H).

¹³C NMR (CD2C12, 150.9 MHZ): δ 13.1 (CH₃), 18.5 (CH₃), 21.1 (CH₃), 21.2 (CH₃), 40.21 (CH₂), 40.22 (CH₂), 56.4 (CH₃), 113.41 (CH), 113.44 (CH), 115.9 (CH₂), 116.7 (C), 117.4 (CH), 117.5 (CH), 119.5 (C), 120.94 (CH), 120.96 (CH), 125.6 (CH), 128.0 (CH), 129.0 (CH), 129.4 (CH), 135.3 (C), 136.06 (C), 136.1 (C), 136.7 (C), 136.8 (C), 137.3 (C), 138.0 (CH), 139.2 (CH), 140.4 (CH), 145.7 (C), 145.8 (C), 150.1 (C), 150.2 (C).

Compound of formula (IV). 1-isopropyl-4-methyl-2-((2-(p-tolyl) prop-1-en-1-yl)oxy)benzene: Thymol (10 g, 66.6 mmol), 1-(1-methoxyprop-1-en-2-yl)-4-methylbenzene (14.0 g, 86.5 mmol) and KHSO₄ (2.72 g, 20 mmol) were charged to a round-bottomed flask equipped with a Vigreux column (175 mm) and distillation head. The mixture was placed under vacuum (14 mbar) and heated at 120° C. (oil bath) for 2 h while distilling out liberated MeOH. The oil bath temperature was increased to 140° C. while the pressure was gradually reduced to 300 mTorr. The mixture was heated an additional 3 h while allowing unreacted starting materials to distill from the mixture. The reaction mixture was diluted with diethyl ether and filtered through a pad of silica gel. After concentrating the filtrate, the remaining residue was subjected to silica gel flash chromatography (hexane:EtOAc 100:0-80:20) followed by bulb-to-bulb distillation (oven 150° C., 20 mTorr) to afford the title compound (4.1 g, 22% yield) as a colorless oil (E/Z=77:23).

¹H NMR (CD₂Cl₂, 500 MHz): δ 1.18z and 1.25; (both d, J=6.9 Hz, 6H), 2.03z and 2.13; (both d, ⁴J=1.4 Hz, 3H), 2.28_{E} and 2.30_{Z} (both s, 3H), 2.32_{Z} and 2.33_{E} (both s, 3H), 3.25_{Z} and 3.28_{E} (both septets, 1H), 6.47_{Z} and 6.82_{E} (both q, ⁴J=1.4 Hz, 1H), 6.79 (s, 0.77H), 6.84 (s, 0.77H), 6.85 (s, 0.46H), 7.10-7.16 (m, 3H), 7.30; and 7.53z (both d, J=8.3 Hz, 2H).

¹³C NMR (CD₂Cl₂, 125.8 MHz): δ 13.1 (CH₃), 18.6 (CH₃), 21.13 (CH₃), 21.15 (CH₃), 21.3 (CH₃), 22.9 (CH₃), 23.0 (CH₃), 27.2 (CH), 27.5 (CH), 116.3 (CH), 116.5 (CH), 117.1 (C), 119.7 (C), 123.8 (CH), 123.9 (CH), 125.5 (CH), 126.5 (CH), 126.7 (CH), 127.9 (CH), 129.0 (CH), 129.5 (CH), 135.07 (C), 135.1 (C), 135.3 (C), 136.7 (C), 136.8 (C), 137.1 (C), 137.3 (C), 138.5 (CH), 139.7 (CH), 155.2 (C), 155.3 (C).

### Example 2

Headspace Analysis from Fabric Softener Application

The compounds prepared in Example 1 (0.075 mmol) were weighed into a vial and dissolved in 0.25 mL of acetone. Unfragranced liquid fabric softener (Table 1, 4.5 g) was added to the vial and the mixture agitated with a Vortex mixer for 1 min. Reference samples were prepared in the same manner using 0.075 mmol of each released volatile. The fabric softener samples were rinsed with deionized water into a 3 L beaker and the beaker was filled to a total volume of 1.5 L. Three, 5-g cotton swatches (ca. 12.5×12.5 cm, weight 270 g/m², item 403 from Testfabrics, West Pittston, PA) were added to the beaker and agitated by hand for 3 min. After an additional 2 min of standing, the swatches were retrieved, and excess water squeezed out by hand. The cloths were hung to dry for either 3 h, 6 h, 17 h or 48 h at room temperature. Three swatches from each time point then were subjected to dynamic headspace analysis.

Each swatch was placed inside a thermostatted (25° C.), headspace sampling cell (about 160 mL volume). Using an air-sampling pump, a constant flow of air (200 mL/min) was drawn through the sampling cell. Prior to entering the sample cell, the air was drawn through a plug of active charcoal and then through a saturated NaCl solution to maintain a constant relative humidity of 75%. Samples were collected for 15 min by directing the air exiting the sampling cell through a cartridge containing 100 mg of Tenax®. When samples were not being collected the air flow was sent through a waste cartridge that also contained 100 mg of Tenax. The air flow was sampled hourly (60-75, 120-135 and 180-195 min) for 15 minutes in addition to an initial sample being collected for the first 15 min.

The cartridges were thermally desorbed with a Gerstel TDU 3.5 with cryofocusing at −30° C. and desorbed into an Agilent 8890 gas chromatograph equipped with an HP1 capillary column (30 m, i.d. 0.25 mm, film 0.25 μm) and coupled with Agilent 5977B mass spectrometer. TDU temperature settings for desorption: 40° C. to 70° C. (30° C./min) hold for 4 min then heat to 260° C. (400° C./min) and hold 5 min. CIS settings (Tenax® packed liner): cryofocus at −30° C. then heat at 12° C./sec to 300° C. and hold for 4 min (heater mode: standard). PTV inlet settings: pressure 7.7 psi, total flow 99 ml/min, septum purge flow 3 ml/min on standard flow mode. The inlet mode was set to solvent vent with a purge flow to split vent set at 95 ml/min and the vent flow at 50 ml/min. The GC oven temperature profile was 52° C. to 110° C. at 20° C./min (hold 2 min) then ramped to 210° C. (20C/min).

The amount of each fragrance volatile collected (reported as ng/L of air) was determined using external standard calibrations of the respective chemicals. At least five acetone solutions were prepared with concentrations of the analytes ranging from 0.05 g/L to 5 g/L. The solutions were injected (0.2 μL) onto Tenax® cartridges and desorbed as described above. Each solution was analyzed in triplicate. Calibration curves were forced through the origin. The average headspace concentration at each time point was determined from the three swatches analyzed and these data were used to determine average headspace concentration over the 3.25 h collection period (Table 2).

TABLE 1
Unfragranced Fabric Softener Composition

| | Unfragranced liquid |
|---|---|
| | fabric softener |
| Ingredient | % Active (w/w) |
| Alkyl quaternary ammonium ester | 7 |
| softening active (Ester Quat) | |
| Free Perfume oil | — |
| Encapsulated perfume | — |
| Formic Acid | 0.045 |
| Hydrochloric acid | 0.0174 |
| Chelant | 0.0007 |
| PEI polymer | 0.0035 |
| Antibacterial active | 0.055 |
| Cross-linked cationic acrylate polymer | 0.12 |
| Antifoam | 0.004 |
| Water | Balance |

TABLE 2
Average dynamic headspace concentrations (ng/L) of perfumery raw materials
obtained from line-dried cotton treated with fabric softener containing
invention's compounds compared to their respective references (ref).

| | | avg headspace conc. (ng/L) after line-drying | | | |
|---|---|---|---|---|---|
| | perfumery raw material | 3 h | 6 h | 17 h | 48 h |
| Cmpd (I) | p-methylacetophenone | 260 (6.9) | 481 (1.1) | 425 (0.5) | 218 (0.3) |
| (ref) | citronellyl formate | 469 (18) | 544 (2.5) | 293 (1.1) | 104 (1.0) |
| | Citronellol | 278 (272) | 173 (18) | 184 (5.1) | 50 (8.6) |
| Cmpd (II) | p-methylacetophenone | 118 (1.5) | 226 (2.0) | 345 (0.9) | 500 (2.4) |
| (ref) | 3,7-dimethyloctan-3-yl | 115 (9.5) | 154 (1.3) | 154 (2.9) | 106 (0.4) |
| | formate | | | | |
| | 3,7-dimethyloctan-3-ol | 125 (0.2) | 124 (0.2) | 224 (0.8) | 249 (1.4) |
| Cmpd (III) | p-methylacetophenone | 81.8 (3.1) | 145 (3.5) | 133 (1.4) | 225 (2.8) |
| (ref) | Eugenol | 40 (148) | 64 (25) | 116 (2.3) | 157 (2.2) |
| Cmpd (IV) | p-methylacetophenone | 41 (7.3) | 100 (1.7) | 267 (1.5) | 373 (6.3) |
| (ref) | Thymol | 37 (626) | 93 (109) | 154 (59) | 211 (3.3) |

These data show that, when applied to cotton fabric from a liquid fabric softener application, the compounds of the invention begin releasing the perfumery ingredients within hours of deposition.

After just 3 h of line-drying, the compounds of formula (I) to (IV) produced more para-methylacetophenone than the reference samples. At most of the time points, the amount of perfumery ingredients released from the invention's compounds was higher than the corresponding reference samples which demonstrates the ability of the invention's compounds to continuously release the perfumery ingredients over an extended period of time in contrast to the reference samples.

### Example 3

Olfactive Evaluation from Fabric Softener

The sensory performance of the four invention's compounds was evaluated on fabric as a function of time. The invention's compounds and their respective controls were dosed onto cotton towels through the wash from a fabric softener formulation.

### Washing and Evaluation Protocol:

A ballast of 30 cotton towel washcloths (12”×12″, de-sized 100% cotton Calderon ITL terries) were washed in a GE Top Loader Model GUD27GSSJ0WW washing machine using the medium wash cycle. 50 grams of unfragranced detergent were added for the wash cycle. 30 grams of fabric softener (Table 1) containing 0.2 wt % actives were added for the rinse cycle. The fabric softeners were prepared by dosing 60 mg of an invention's compound into 29.94 g of unfragranced fabric softener. Their respective controls were prepared by dosing 27 mg of para-methylacetophenone and 33 mg of the expected alcohol or phenol into 29.94 g of the unfragranced fabric softener. Washed towels were then hung on drying racks for sensory evaluation over time. The towels were evaluated for odor intensity wet at to, half dry at t=6 hours, dry at t=12 hours and t=24 hours by n=6 expert panelists. At to all the control towels had a stronger olfactive intensity than the corresponding invention's compound-treated towels. After 6 hours of drying and thereafter, Compounds (I), (II) and (III) provided higher olfactive intensities than the corresponding controls. Compound (IV) also delivered the expected aroma during the drying process and delivered a higher olfactive intensity than the control after 24 hours.

TABLE 3
Average olfactive intensity values of line-dried cotton towels treated with fabric softener
containing invention's compounds compared to their respective control towels as a function
of drying time from t₀ to t = 24 hours. Olfactive intensity evaluated on a 10-point
intensity scale (1 = no odor, 10 = very strong odor) by n = 6 expert panelists.

| | | Intensity Scores (1-10) | | | | | | | |
|---|---|---|---|---|---|---|---|---|---|
| | | | | | | Cmpd | Cmpd | Cmpd | Cmpd |
| | | Cmpd | Cmpd | Cmpd | Cmpd | (I) | (II) | (III) | (IV) |
| Time | Sample | (I) | (II) | (III) | (IV) | Control | Control | Control | Control |
| 0 | Wet Fabric 0 hr | 1.7 | 2.5 | 3.3 | 4.8 | 5.7 | 4.3 | 5.8 | 7.3 |
| 0 | Stdev | 0.8 | 0.8 | 1.4 | 1.9 | 1.4 | 1.4 | 2.3 | 2.4 |
| 6 | Half-Dry Fabric | 4.0 | 4.0 | 4.6 | 4.7 | 3.5 | 2.0 | 3.8 | 5.8 |
| | 6 hrs | | | | | | | | |
| 6 | Stdev | 2.3 | 1.9 | 0.5 | 0.8 | 1.6 | 1.3 | 2.3 | 1.4 |
| 12 | Dry Fabric 12 | 2.3 | 2.5 | 4.8 | 3.5 | 0.5 | 0.5 | 4.3 | 4.3 |
| | hrs | | | | | | | | |
| 12 | Stdev | 1.1 | 0.7 | 0.4 | 0.7 | 0.7 | 0.7 | 0.4 | 3.2 |
| 24 | Dry Fabric 24 | 2.8 | 3.4 | 3.3 | 5.1 | 1.9 | 1.0 | 1.5 | 2.8 |
| | hrs | | | | | | | | |
| 24 | Stdev | 1.3 | 1.4 | 1.0 | 0.9 | 0.9 | 0.9 | 0.4 | 1.5 |

### Example 4

Olfactive Evaluation of Dry Towels from Fabric Softener after Rewetting

Invention's compounds exhibit extended release of perfuming ingredients at 24 hours, with enhanced impact after rewetting of fully dried garments, which simulates performance after perspiration, exposure to humidity, and wetting of dried or stored garments at longer times.

Cotton towels were washed and line-dried for 24 h as described in Example 3. To rewet the towels, dried washcloth towels were sprayed 5 times with room temperature water using a mister bottle to evenly dampen the fabric and then were left 2 minutes before sensory evaluation.

TABLE 4
Average olfactive intensity values of perfumery raw materials released from
line-dried cotton towels treated with fabric softener containing invention's
compounds and line-dried for 24 hours. Dried washcloth towels were evaluated
before and after rewetting with 5 sprays of room temperature water from a
mister bottle. Olfactive intensity evaluated on a 10-point intensity scale
(1 = no odor, 10 = very strong odor) by n = 6 expert panelists.

| | | Intensity Scores (1-10) | | | |
|---|---|---|---|---|---|
| | | Compound | Compound | Compound | Compound |
| Time | Sample | (I) | (II) | (III) | (IV) |
| 24 | Dry Fabric 24 hrs | 2.8 | 3.4 | 3.3 | 5.1 |
| 24 | Stdev | 1.3 | 1.4 | 1.0 | 0.9 |
| 24 | Dry Fabric 24 hrs | 4.4 | 5.0 | 4.7 | 5.3 |
| | rewet | | | | |
| 24 | Stdev | 1.6 | 1.3 | 1.3 | 1.5 |

### Example 5

Olfactive Evaluation from Fabric Softener Against 4-Methyl-3-Hexenoic Acid (4M3HA)

A model malodor counteraction (MOC) test method was developed to measure the ability of an ingredient to counteract the smell of a malodor on fabric. The malodor control efficacy was tested through the wash from the four invention's compounds and their respective control mixtures (para-methylacetophenone+perfumery alcohol/phenol) against 4-methyl-3-hexenoic acid (4M3HA, E/Z=70:30), a synthetic malodor compound which was validated as an appropriate surrogate for the characteristic malodor formed during slow indoor line-drying in humid environments (K. Takeuchi, et. al. Flavour and Fragrance Journal, 2012, vol. 27, pages 89-94).

### Washing, 4M3HA Application and Malodor Counteraction Evaluation Protocol:

A ballast of 30 cotton towel washcloths (12″×12″, de-sized 100% cotton Calderon ITL terries) were washed in a GE Top Loader Model GUD27GSSJ0WW washing machine using the medium wash cycle. 50 grams of unfragranced detergent were added for the wash cycle. 30 grams of fabric softener (Table 1) containing 0.2 wt % actives were added for the rinse cycle. The fabric softeners were prepared by dosing 60 mg of an invention's compound into 29.94 g of unfragranced fabric softener. Their respective controls were prepared by dosing 27 mg of para-methylacetophenone and 33 mg of the expected alcohol or phenol into 29.94 g of the unfragranced fabric softener. Washed towels were then hung on drying racks to partially dry for 6 hours before wrapping in aluminum foil on one side and the edges, and folding in half to form a booklet-style pouch with foil on the outside. The foil pouch can be opened to expose a rectangle of fabric.

A model malodor solution of 5.0 wt % 4-methyl-3-hexenoic acid (4M3HA) was prepared in triethyl citrate, and 100 μL of the malodor solution was pipetted in the center of each cotton wash cloth previously treated with fabric softener containing the invention's compounds or their respective controls. The centers of the towels were marked with an ink dot to orient panelists. The aluminum foil-wrapped pouches were folded shut and allowed to equilibrate for 20-30 min before bringing to the evaluation room for malodor counteraction evaluations. Malodor reference towels were prepared following the same procedure but using unfragranced fabric softener. After the six-hour evaluation, the foil pouches were left open to allow the towels to dry before being evaluated again 24 hours after removal from the washing machine.

Panelists first smelled the identified malodor reference to become familiar with the character and intensity of the malodor. Panelists then evaluated the treated towel for malodor intensity compared to the malodor reference by opening the foil pouch and smelling in the center. There was a two-minute test period between sample evaluations, and evaluation order was randomized.

Additionally, the effect of rewetting on counteraction was assessed for the towels evaluated at 24 hours. The foil wrapped towels were sprayed 5 times with room temperature water from a mister bottle. The foil pouches were folded shut and the towels left for 2 minutes before evaluating.

The ability of an active to counteract or enhance the reference malodor was assessed using an established scale. On the malodor counteraction scale, the reference malodor scores zero and positive values indicate perceived malodor counteraction.

TABLE 5
Scoring for malodor counteraction sensory evaluation protocol
relative to a reference malodor (reference malodor score = 0).

| Score | Malodor Counteraction |
|---|---|
| 3 | Very good counteracting of the malodor |
| 2 | Good counteracting |
| 1 | Slight counteracting |
| 0 | No perceived effect |
| −1 | Possible enhancement of the malodor |
| −2 | Clearly enhancing the malodor |

TABLE 6
Malodor counteraction data for the invention's compounds and corresponding controls
versus 4M3HA model malodor dosed onto cotton towel washcloths treated with fabric
softener. Reference 4M3HA-treated towel scores zero on malodor counteraction
scale (−2 = clearly enhancing the malodor; −1 = possible enhancement
of the malodor; 0 = no perceived effect; 1 = slight counteracting;
2 = good counteracting; 3 = very good counteracting of the malodor). n = 6 expert panelists.

| | | Malodor Counteraction Scores (−2 to 3) | | | | | | | |
|---|---|---|---|---|---|---|---|---|---|
| | | | | | | Cmpd | Cmpd | Cmpd | Cmpd |
| Time | | Cmpd | Cmpd | Cmpd | Cmpd | (I) | (II) | (III) | (IV) |
| (hrs) | Sample | (I) | (II) | (III) | (IV) | Control | Control | Control | Control |
| 6 | 6 hr Avg | 1.7 | 2.0 | 1.9 | 2.0 | 0.8 | 1.2 | 2.1 | 1.8 |
| 6 | Stdev | 0.4 | 0.8 | 1.1 | 0.6 | 0.6 | 0.8 | 0.2 | 0.5 |
| 24 | 24 hr Avg | 1.7 | 2.2 | 2.1 | 1.7 | 0.6 | 1.2 | 0.7 | 1.1 |
| 24 | Stdev | 0.6 | 0.4 | 0.8 | 0.4 | 1.1 | 0.6 | 0.8 | 0.9 |
| 24 | 24 hr rewet | 2.1 | 2.8 | 2.3 | 2.2 | 1.1 | 1.3 | 1.4 | 1.5 |
| | Avg | | | | | | | | |
| 24 | Stdev | 1.2 | 0.3 | 0.8 | 0.4 | 0.4 | 0.8 | 0.9 | 0.4 |

These olfactive evaluations showed that the invention's compounds provided the desired malodor counteraction against 4M3HA. They provided better or similar malodor counteraction for the 6-hour samples relative to the controls. All the invention's compounds were perceived as delivering better malodor counteraction for both the dry and rewet 24-hour samples.

### Example 6

Olfactive Evaluation from Fragranced Market Product Fabric Softener Against 4-Methyl-3-Hexenoic Acid (4M3HA)

The malodor counteraction performance of the invention's compounds against a model malodor compound 4M3HA was assessed from a fragranced market product.

A ballast of 30 cotton towel washcloths (12″×12″ de-sized 100% cotton Calderon ITL terries) were washed in a GE Top Loader Model GUD27GSSJ0WW washing machine using the medium wash cycle.

50 grams of unfragranced detergent were added for the wash cycle. 30 grams of fragranced liquid fabric softener (Table 7) containing 0.2 wt % actives were added for the rinse cycle. The fabric softeners were prepared by dosing 60 mg of an invention's compound into 29.94 g of the fragranced liquid fabric softener.

Washed towels were then hung on drying racks to partially dry for 6 hours before wrapping in aluminum foil to form a booklet-style pouch. A model malodor solution of 5.0 wt % 4-methyl-3-hexenoic acid (4M3HA) was prepared in triethyl citrate, and 100 μL of the malodor solution was pipetted in the center of each cotton washcloth. The centers of the towels were marked with an ink dot to orient panelists. The aluminum foil-wrapped pouches were folded shut and allowed to equilibrate for 20-30 min before bringing to the evaluation room for malodor counteraction evaluations. Malodor reference towels were prepared following the same procedure but using unfragranced fabric softener. Sensory evaluations for malodor counteraction were conducted and scored as described in Example 5.

TABLE 7
Fragranced Liquid Fabric Softener Composition

| | Fragranced liquid |
|---|---|
| | fabric softener |
| Ingredient | % Active (w/w) |
| Alkyl quaternary ammonium ester | 7 |
| hsoftening active (Ester Quat) | |
| Free Perfume oil | 1.5 |
| Encapsulated perfume | 0.30 |
| Formic Acid | 0.045 |
| Hydrochloric acid | 0.0174 |
| Chelant | 0.0007 |
| PEI polymer | 0.0035 |
| Antibacterial active | 0.055 |
| Cross-linked cationic acrylate polymer | 0.12 |
| Antifoam | 0.004 |
| Water | Balance |

TABLE 8
Malodor counteraction data for the invention's compounds versus 4M3HA model malodor
dosed onto cotton towel washcloths treated with the Fragranced Liquid Fabric Softener
as reported in Table 7. Reference 4M3HA-treated blank towel scores zero on malodor
counteraction scale (−2 = clearly enhancing the malodor; −1 = possible
enhancement of the malodor; 0 = no perceived effect; 1 = slight counteracting;
2 = good counteracting; 3 = very good counteracting of the malodor). n = 7 panelists.

| | | | Fragranced | Fragranced | Fragranced | Fragranced |
|---|---|---|---|---|---|---|
| | | Fragranced | liquid | liquid | liquid | liquid |
| | | liquid | fabric | fabric | fabric | fabric |
| Time | | fabric | softener + | softener + | softener + | softener + |
| (hrs) | Sample | softener | Cmpd (I) | Cmpd (II) | Cmpd (III) | Cmpd (IV) |
| 6 | 6 hr Avg | 1.6 | 2.2 | 2.5 | 2.5 | 2.3 |
| 6 | Stdev | 0.4 | 0.5 | 0.3 | 0.8 | 0.3 |

These olfactive evaluations showed that the fragranced market product did provide a malodor counteraction benefit but that the invention's compounds increased the malodor counteraction efficacy against 4M3HA when added to a perfumed fabric softener.

### Example 7

Olfactive Evaluation from Unscented Fabric Softener with Consumer Garments Exhibiting Characteristic Indoor-Drying Malodor

The malodor counteraction performance of the invention's compounds when applied from a liquid fabric softener onto consumer garments exhibiting characteristic indoor-drying malodor was assessed in a miniaturized laundry assay. Fabrics sourced from Japanese consumers reporting indoor-drying malodor were sorted by fabric type, malodor character and malodor intensity. Additional criteria for fabric volunteers included age of washing machine, chronic indoor drying malodor complaints and living in a smoke-free home. Cotton towels with similar malodor character and intensity were selected by experts, cut into strips, sorted to have a representative mix for each laundry batch and washed in a mesh bag with a 2 kg cotton fabric ballast. They were washed in a GE Top Loader Model GUD27GSSJ0WW washing machine using the medium wash cycle. 50 grams of unfragranced HDL liquid detergent were added for the wash cycle. 30 grams of unscented fabric softener (Table 1) containing 0.2 wt % actives were added for the rinse cycle. The fabric softeners were prepared by dosing 60 mg of an invention's compound into 29.94 g of unfragranced fabric softener.

The fabric strips were then placed in sealed glass jars (4-6 strips weighing 8-10 grams total per 16 oz jar) to incubate in an oven set to 30° C. for 5 hours prior to evaluation. The jar lids were opened for 45 minutes, and then closed for 15 minutes prior to olfactive evaluation using the jar protocol. Following evaluation at 6 hours, the strips were carefully removed from the jars and set on foil trays to dry under ambient/RT conditions for 18 hours, before being returned to the jars for evaluation at the 24-hour time point. For the rewetting trial, the strips were misted with 5 sprays of water, gently agitated in the jar, and were evaluated after 15 minutes. Malodor reference strips were prepared following the same procedure but using unfragranced fabric softener. Sensory evaluations for malodor counteraction were conducted and scored as described in Example 5.

TABLE 9
Malodor counteraction data for the invention's compounds on fabric
strips exhibiting characteristic indoor-drying malodor from an unscented
fabric softener in a jar test at 6 hours and 24 hours with and without
rewetting. Malodor reference is fabric strips treated with unscented
fabric softener with no added active. Reference fabric strips in jar
test score zero on malodor counteraction scale (0 = no perceived
effect; 1 = slight counteracting; 2 = good counteracting;
3 = very good counteracting of the malodor). n = 8 expert panelists.

| | | | Cmpd | Cmpd | Cmpd | Cmpd |
|---|---|---|---|---|---|---|
| Time | | Sample | (I) | (II) | (III) | (IV) |
| 6 | hrs | 6 hr Avg | 1.6 | 1.4 | 2.3 | 2.0 |
| 6 | hrs | Stdev | 0.6 | 0.5 | 0.2 | 0.5 |
| 24 | hrs | 24 hr Avg | 2.2 | 2.3 | 2.9 | 2.9 |
| 24 | hrs | Stdev | 0.5 | 0.6 | 0.2 | 0.2 |
| 24 | hrs | 24 hr rewet Avg | 1.9 | 0.6 | 2.2 | 1.8 |
| 24 | hrs | Stdev | 0.6 | 0.8 | 0.2 | 0.7 |

These olfactive evaluations showed that the invention's compounds provided the desired malodor counteraction when applied to consumer garments exhibiting the characteristic indoor line-drying malodor at 6 and 24 hours. The perceived malodor strength of the untreated reference control was noted prior to the evaluations to be strong at 6 hours, weak at 24 hours and moderate at 24 hours after rewetting with water. The invention's compounds work very well to counteract the malodor at 6 and 24 hours, and remain effective in spite of the increase in malodor intensity after rewetting the dried fabric at 24 hours.

### Example 8

Olfactive Evaluation from Market Product Fabric Enhancer with Consumer Garments

The malodor counteraction performance of the invention's compounds when applied from a fragranced product liquid fabric softener (Table 7) onto consumer garments exhibiting characteristic indoor-drying malodor was assessed in a miniaturized laundry assay. Fabrics sourced from Japanese consumers reporting indoor-drying malodor were sorted by fabric type, malodor character and malodor intensity. Additional criteria for fabric volunteers included age of washing machine, chronic indoor drying malodor complaints and living in a smoke-free home. Cotton towels with similar malodor character and intensity were selected by experts, cut into strips, sorted to have a representative mix for each laundry batch and washed in a mesh bag with a 2 kg cotton fabric ballast. They were washed in a GE Top Loader Model GUD27GSSJ0WW washing machine using the medium wash cycle. 50 grams of unfragranced HDL liquid detergent were added for the wash cycle. 30 grams of unscented fabric softener containing 0.2 wt % actives were added for the rinse cycle. The fabric softeners were prepared by dosing 60 mg of an invention's compound into 29.94 g of unfragranced fabric softener. Malodor reference is fabric strips treated with unscented fabric softener with no added active.

The fabric strips were then placed in sealed glass jars (4-6 strips weighing 8-10 grams total per 16 oz jar) to incubate in an oven set to 30° C. for 5 hours prior to evaluation. The jar lids were opened for 45 minutes, and then closed for 15 minutes prior to olfactive evaluation using the jar protocol. Following evaluation at 6 hours, the strips were carefully removed from the jars and set on foil trays to dry under ambient/RT conditions for 18 hours, before being placed in the jars for evaluation at the 24-hour time point. For the rewetting trial, the strips were misted with 5 sprays of water, gently agitated in the jar with the lid closed, and were evaluated after 15 minutes. Sensory evaluations for malodor counteraction were conducted and scored as described in Example 5.

TABLE 10
Malodor counteraction data for the invention's compounds on fabric
strips exhibiting characteristic indoor-drying malodor from a market
product fabric softener (MP) in a jar test evaluated out of the machine
(t₀), at 6 hours and at 24 hours before and after rewetting. Malodor
reference is fabric strips treated with unscented fabric softener
with no added active. Reference fabric strips in jar test score
zero on malodor counteraction scale (0 = no perceived effect;
1 = slight counteracting; 2 = good counteracting; 3 =
very good counteracting of the malodor). n = 8 expert panelists.

| | | MP + | MP + | MP + | MP + |
|---|---|---|---|---|---|
| | | Cmpd | Cmpd | Cmpd | Cmpd |
| Sample Time | MP | (I) | (II) | (III) | (IV) |
| t₀ Fresh Avg | 2.3 | 2.5 | 2.7 | 2.8 | 2.6 |
| t₀ Fresh Stdev | 0.3 | 0.5 | 0.3 | 0.3 | 0.5 |
| 6 hr Avg | 1.1 | 1.7 | 1.5 | 1.7 | 1.7 |
| 6 hr Stdev | 0.2 | 0.5 | 0.6 | 0.2 | 0.6 |
| 24 hr Avg | 1.8 | 2.8 | 2.9 | 2.9 | 2.9 |
| 24 hr Stdev | 1.7 | 0.2 | 0.2 | 0.2 | 0.2 |
| 24 hr rewet Avg | 0.9 | 1.7 | 2.1 | 1.7 | 1.4 |
| 24 hr rewet Stdev | 0.4 | 0.5 | 0.5 | 0.7 | 0.8 |

These olfactive evaluations showed that the invention's compounds provided significant malodor counteraction when applied to consumer garments exhibiting the characteristic indoor line-drying malodor and outperform the scented market product at all times ranging from fresh in the jar to 6 hours, 24 hours and 24 hour after rewetting. The perceived malodor strength of the untreated reference control (unscented fabric softener as reported in Table 1) was noted prior to the evaluations to be strong at 6 hours, weak at 24 hours and strong again at 24 hours after rewetting with water. The invention's compounds work very well to counteract the malodor at 6 and 24 hours and remain effective in spite of the significant increase in malodor intensity after rewetting the dried fabric at 24 hours.

### Example 9

### Olfactive Evaluation of Para-Methylacetophenone and Acetophenone Against Malodor 4-Methyl-3-Hexenoic Acid (4M3HA)

A malodor counteraction (MOC) test was used to compare the ability of para-methylacetophenone and acetophenone to counteract the smell of a malodor on fabric. A synthetic malodor compound, 4-methyl-3-hexenoic acid (4M3HA, E/Z=70:30), was used as an appropriate surrogate for the characteristic malodor formed during slow indoor line-drying in humid environments (K. Takeuchi, et. al. Flavour and Fragrance Journal, 2012, vol. 27, pages 89-94).

A ballast of 30 cotton towel washcloths (12″×12″, de-sized 100% cotton Calderon ITL terries) were washed in a GE Top Loader Model GUD27GSSJ0WW washing machine using the medium wash cycle and 50 grams of unfragranced detergent. The towels were machine dried. Solutions of para-methylacetophenone or acetophenone at 1.0 wt % in dipropylene glycol were prepared, and 34 μL of each was diluted in 30 g of DI water. Each 30 g aqueous solution was evenly distributed over separate cotton towels using a pipette. The towels were wrapped in aluminum foil to form booklet-style pouches that were folded in half to prevent water evaporation from the towels. After equilibrating for 4 hours, the pouches were unfolded, and the center of the towels marked with an ink dot to orient panelists. A model malodor solution of 5.0 wt % 4-methyl-3-hexenoic acid (4M3HA) in triethyl citrate (100 μL) was pipetted in the center of each towel. The aluminum foil pouches were folded shut and the towels allowed to equilibrate for 20-30 min before bringing to the evaluation room for malodor counteraction evaluations. Reference malodor towels were prepared in the same manner except that the towels were only treated with 30 g of DI water and 100 μL of the 4M3HA solution.

Panelists first smelled the identified malodor reference to become familiar with the character and intensity of the malodor. Panelists then evaluated the treated towels for malodor intensity compared to the malodor reference by opening the foil pouch and smelling in the center. The evaluation order was randomized among the panelists.

TABLE 11
Malodor counteraction data for para-methylacetophenone or
acetophenone versus 4M3HA model malodor dosed onto cotton
towel washcloths. 4M3HA-treated towel scores zero on malodor
counteraction scale (−2 = clearly enhancing the malodor; −1 =
possible enhancement of the malodor; 0 = no perceived
effect; 1 = slight counteracting; 2 = good
counteracting; 3 = very good counteracting of the
malodor). n = 3 expert panelists.

| Active | Malodor Counteraction Score (−2 to 3) |
|---|---|
| Acetophenone | 1.33 |
| Para-methylacetophenone | 2 |

These olfactive evaluations show that para-methylacetophenone provides greater malodor counteraction against 4M3HA. Therefore, there is an advantage to using a malodor counteracting technology that releases para-methylacetophenone for use against moldy malodor generated on laundry.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A consumer product comprising a compound selected from the group consisting of: a compound of formula (I), a compound of formula (II), a compound of formula (III), a compound of formula (IV), and mixtures thereof 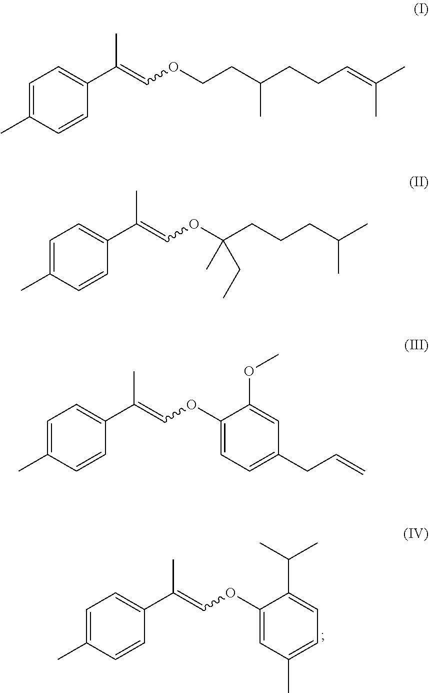
or in the form of a stereoisomer of the compound of formula (I), a compound of formula (II), a compound of formula (III), a compound of formula (IV), and mixtures thereof.

2. The consumer product according to claim 1, wherein the double bond of the compounds of formula (I) to (IV) is in E configuration.

3. The consumer product according to claim 1, wherein the compound is 1-(1-((3,7-dimethyloct-6-en-1-yl)oxy) prop-1-en-2-yl)-4-methylbenzene or (E)-1-(1-((3,7-dimethyloct-6-en-1-yl)oxy) prop-1-en-2-yl)-4-methylbenzene.

4. The consumer product according to claim 1, wherein the compound is 4-allyl-2-methoxy-1-((2-(p-tolyl) prop-1-en-1-yl)oxy)benzene or (E)-4-allyl-2-methoxy-1-((2-(p-tolyl) prop-1-en-1-yl)oxy)benzene.

5. The consumer product according to claim 1, wherein the consumer product comprises, based on total weight of the consumer product, from about 0.0001% to about 25% of the compound selected from the group consisting of a compound of formula (I), a compound of formula (II), a compound of formula (III), a compound of formula (IV) and mixtures thereof.

6. The consumer product according to claim 1, wherein the consumer product is a fabric and/or hard surface cleaning and/or treatment composition, said composition comprising, based on total composition weight, from about 0.00001% to about 25% of the compound selected from the group consisting of a compound of formula (I), a compound of formula (II), a compound of formula (III), a compound of formula (IV) and mixtures thereof.

7. The consumer product according to claim 1, wherein the consumer product is a detergent, said detergent comprising, based on total detergent weight, from about 0.00001% to about 25% of the compound selected from the group consisting of a compound of formula (I), a compound of formula (II), a compound of formula (III), a compound of formula (IV) and mixtures thereof.

8. The consumer product according to claim 1, wherein the consumer product is a highly compacted consumer product, said highly compacted consumer product comprising, based on total highly compacted consumer product weight, from about 0.00001% to about 25% of the compound selected from the group consisting of a compound of formula (I), a compound of formula (II), a compound of formula (III), a compound of formula (IV) and mixtures thereof.

9. A method of treating an area with the consumer product of claim 1.

10. A method of treating an area with the consumer product of claim 3.

11. A method of treating an area with the consumer product of claim 4.

12. A method of treating a fabric or hard surface with the consumer product of claim 6.

13. The method of claim 12, wherein the compound is 1-(1-((3,7-dimethyloct-6-en-1-yl)oxy) prop-1-en-2-yl)-4-methylbenzene or (E)-1-(1-((3,7-dimethyloct-6-en-1-yl)oxy) prop-1-en-2-yl)-4-methylbenzene.

14. The method of claim 12, wherein the compound is 4-allyl-2-methoxy-1-((2-(p-tolyl) prop-1-en-1-yl)oxy)benzene or (E)-4-allyl-2-methoxy-1-((2-(p-tolyl) prop-1-en-1-yl)oxy)benzene.

15. A method of treating a fabric with the consumer product of claim 7.

16. The method of claim 15, wherein the compound is 1-(1-((3,7-dimethyloct-6-en-1-yl)oxy) prop-1-en-2-yl)-4-methylbenzene or (E)-1-(1-((3,7-dimethyloct-6-en-1-yl)oxy) prop-1-en-2-yl)-4-methylbenzene.

17. The method of claim 15, wherein the compound is 4-allyl-2-methoxy-1-((2-(p-tolyl) prop-1-en-1-yl)oxy)benzene or (E)-4-allyl-2-methoxy-1-((2-(p-tolyl) prop-1-en-1-yl)oxy)benzene.
